(19) United States
(12) Patent Application Publication
(10) Pub. No.: US 2012/0283318 A1
(43) Pub. Date: Nov. 8, 2012

(54) REPLICATING VIRAL VECTORS FOR GENE THERAPY

(76) Inventors: Ya-Fang Mei, Umea (SE); Goran Wadell, Umea (SE)

(21) Appl. No.: 13/500,300

(22) PCT Filed: Oct. 4, 2010
(86) PCT No.: PCT/SE2010/051066

§ 371 Date: Jul. 23, 2012

Related U.S. Application Data
(60) Provisional application No. 61/248,516, filed on Oct. 5, 2009.

(51) Int. Cl. A61K 31/713; A61P 35/00; C12N 5/10; C12P 21/00; A61P 31/12; A61N 5/10; A61P 9/00; A61P 1/16; A61P 33/00; A61P 31/16; A61P 31/14; C12N 15/861; A61P 31/04

(52) U.S. Cl. 514/44 R; 435/320.1; 435/325; 435/69.1; 600/1

(57) ABSTRACT

The present invention concerns the field of gene therapy and in particular the use of specific adenoviral vector systems for gene therapy, said vector systems offering enhanced efficiency and specificity for gene delivery. More specifically, the present invention provides replicating-competent adenoviral vector systems carrying one or more inserted heterologous gene. The adenoviral vectors system according to the invention are characterized by high binding efficiency and infectivity to cells of neural origin, endothelial cells, carcinoma cells and dendritic cells.

### FIELD OF THE INVENTION

The present invention concerns the field of gene therapy and in particular the use of specific adenoviral vector systems for gene therapy, said vector systems offering enhanced efficiency and specificity for gene delivery. More specifically, the present invention also provides replicating-competent adenoviral vector systems carrying one or more inserted heterologous gene. The adenoviral vectors system according to the invention are characterized by high binding efficiency and infectivity to cells of neural origin, endothelial cells, carcinoma cells and dendritic cells.

### BACKGROUND OF THE INVENTION

Gene transfer into neural cells has grown into a big field in neuroscience. The usage of gene transfer is ranging from treatment of genetic diseases, tumours and acquired degenerative encephalopaties such as Alzheimer's disease and Parkinson's disease to being a powerful tool in the study of biological mechanisms. An important application of gene transfer is gene therapy, which is when a therapeutic gene is inserted into the cells by ex vivo or in vivo techniques. One of the obstacles to overcome with gene therapy is to get the gene into the right cell type. The choice of cell depends on the nature of the disease. In diseases such as Haemophilia B where a blood clotting factor (IX) is missing in plasma, it is not as important to reach the damaged cell type. Even if the liver normally makes the clotting factor, it does not matter if the therapeutic gene is inserted in muscle cells, fibroblasts or even blood cells as long as the clotting protein is produced in therapeutic amounts and with the correct post-translational modifications. The protein accessibility to its target and the immunological status is also important. A protein that is normally expressed only inside the blood-brain barrier could for example be immunogenic if exposed on the outside. Other emerging areas where gene transfer can become an important therapeutic tool are regenerative and reparative medicine.

There are two different approaches to deliver the DNA (target genes) into the cells. The first is the usage of non-viral vectors to insert the DNA. The non-viral approach consists of methods like direct injection of the DNA, mixing the DNA with polylysine or cationic lipids that allow the DNA to be internalised. Most of these approaches have a low efficiency of delivery and transient expression of the gene. The second and more widely used approach to insert the DNA is by using viral vectors. Viruses have evolved a mechanism to insert their DNA into cells very effectively, but the side effect is that humans have evolved an effective immune response to eliminate viruses from the body.

To function as a viral vector in the nervous system, the vector should have certain properties. Since almost all cells in the brain are non-dividing, the vector must be able to infect non-dividing cells. A good vector also needs to be non-toxic to the cells in the dose required for infection (direct cytotoxicity i.e. by capsid proteins and antigenicity). After the DNA has entered the nucleus it should integrate in a site-specific location in the host chromosome or become a stable extra-chromosomal element (episome). The desired gene should be expressed without interfering with the cellular expression machinery.

Viral vectors used for gene delivery into the nervous system are herpes simplex-1 virus, adenovirus, adeno-associated virus, complex retrovirus, and simple retrovirus such as lentivirus. For this purpose the viruses pathogenicity genes have been deleted and their ability to replicate has-been incapacitated.

Adenoviruses are good candidates for gene therapy towards the nervous system for a number of reasons. They can infect both dividing and non-dividing cells, the viral genome is relatively stable and is easy to manipulate by recombinant DNA techniques. Replication of the virus is efficient in permissive cells and the pathogenicity is low. One of the obstacles to overcome with all viral vectors is to achieve a sustained expression. The viral vector evokes an innate immune response and an adaptive immune response that is both cell mediated and humoral and the infected cells may become destroyed by the inflammatory response within a couple of weeks. The immune response evoked by adenoviral gene transfer is however different in the brain compared to in the peripheral tissues. The immune response in the brain is not sufficiently strong to eradicate the adenovirus infected cells. However, if the individual has had a previous exposure to the adenovirus or was inoculated with the virus later, a strong inflammatory response can be evoked also in the brain. Consequently, adenovirus serotypes of low prevalence in the society should preferentially be used as vectors to be more successful as gene delivery vectors.

The adenoviruses are a family of DNA viruses that can infect both dividing and non-dividing cells. They do not usually integrate into the host chromosome, instead they are replicated as extra-chromosomal elements inside the nucleus of the host cell. Adenoviruses can bind to a range of different cell types. The clinical picture of an adenovirus infection is often respiratory infection or gastro-enteritis. A tonsillitis similar to a streptococcus A infection is also not uncommon. Some of the adenovirus serotypes can cause epidemic keratoconjunctivitis or in some cases even meningitis or encephalitis.

There are at the present 51 known serotypes of adenovirus, which have been divided into six different subgenera, A-F, depending on their biological properties and genetic homology. Virus within the same subgenus shares more than 50% DNA homology whereas viruses in different subgenera have less than 20% homology (Wadell G., Adenoviruses (adenoviridae): General features. Encyclopaedia of Virology. Ed. Webster R. G., Granoff A., Academic Press Ltd London, pp 1-7, 1999).

Human adenoviruses are non-enveloped and about 80 nm in diameter with a 36 Kbp double stranded DNA. The virion capsid is composed of 240 hexon capsomers and 12 vertex capsomers. An antenna-like fibre projects from each vertex capsomer (located at the corners of the icosahedral capsid). The epitopes capable of making serotype specific antibodies and hence also the epitopes determining the serotype are located on the external portions of the hexons and on the most distal knob of the fibre.

Infection starts with the attachment of the fibre knob to a cellular receptor on the permissive cell. The cellular receptor for the virus fibre is coxsackievirus-adenovirus receptor (CAR) for all subgenera except subgenus B. Additional cellular receptors for adenoviruses are CD46, CD80, CD86, sBAR and sialic acid. The viral penton base then binds to the cellular integrin αᵥβ₃ and the virus is internalised by endocytosis into an endosome. Upon fusion with a lysosome, the pH is lowered leading to alterations in the viral capsid, releasing the virion from the endo-lysosome. The virion is then transported to the nucleus where the replication and transcription takes place. The spliced mRNA is translated in the cytoplasm. Production of the fibre protein can be detected 9-11 h after infection. The structural proteins are then translocated into the nucleus where assembly of new virions takes place.

Efficient adenovirus infection of target cells as described above depends upon the presence of the coxsackie-adenovirus cell surface receptor, CAR, which is the primary receptor for all human adenoviruses except species B viruses. Several human carcinoma cells lacking CAR have been reported. A lower level of CAR expression has been demonstrated in human bladder cancer cells (Li et al. 1999. Loss of adenoviral receptor expression in human bladder cancer cells: a potential impact on the efficacy of gene therapy. Cancer Res 59(2), 325-30). Prostate cancer specimens with a low-grade Gleason score (GS) and with a high-grade GS or malignant cells showed a similar membrane staining pattern to that of normal tissue, but CAR-specific staining was markedly diminished in high-grade GS specimens (Rauen et al. 2002. Expression of the coxsackie adenovirus receptor in normal prostate and in primary and metastatic prostate carcinoma: potential relevance to gene therapy. Cancer Res 62(13), 3812-8). Thus, the amount of CAR expression in prostate cancer indicated the patient's prognosis. In a mouse model, transfection with CAR increased the efficacy of Ad5 vectors (Bao et al. 2005. Human coxsackie adenovirus receptor (CAR) expression in trans-genic mouse prostate tumors enhances adenoviral delivery of genes. Prostate 64(4), 401-7).

The use of adenovirus vectors to control metastasis of cancer has been hampered by the low expression of the CAR receptor. Furthermore, the high sero-prevalence of this adenovirus type in the human population induces adverse reactions in patients. To increase transduction efficacy, Ad5 vectors modified to carry the fiber of a species B adenovirus, or the integrin binding site of RGD or the laminin-derived peptid SIKVAV in their fiber knobs have been successfully used in several cases (Rajecki et al. 2007. Treatment of prostate cancer with Ad5/3Delta24hCG allows non-invasive detection of the magnitude and persistence of virus replication in vivo. Mol Cancer Ther 6(2), 742-51; Stevenson et al. 2007, Incorporation of a laminin-derived peptide (SIKVAV) on polymer-modified adenovirus permits tumor-specific targeting via alpha6-integrins. Cancer Gene Ther 14(4), 335-45). However, the preexisting immunity against Ad5 diminishes the efficacy of the vector and an alternative adenovirus vector that uses a receptor other than CAR represents an attractive option.

Ad11, belonging to species B adenoviruses, uses CD46 and sBAR as the primary receptors (Segerman et al. 2003, There are two different species B adenovirus receptors: sBAR, common to species B1 and B2 adenoviruses, and sB2AR, exclusively used by species B2 adenoviruses. J Virol 77(2), 1157-62; Tuve et al. 2006, A new group B adenovirus receptor is expressed at high levels on human stem and tumor cells. J Virol 80(24), 12109-20). CD46 is upregulated on the surface of tumour cells. An additional benefit of using Ad11 as a vector is the low seroprevalence in humans. Replication-incompetent species B vectors have been reported (Lemckert et al. 2005. Immunogenicity of heterologous prime-boost regimens involving recombinant adenovirus serotype 11 (Ad11) and Ad35 vaccine vectors in the presence of anti-ad5 immunity. J Virol 79(15), 9694-701; Stone et al. 2005. Development and assessment of human adenovirus type 11 as a gene transfer vector. J Virol 79(8), 5090-104) and some limitations of using such vectors have been observed: e.g. the virus can only replicate in its packaging cells and it cannot spread to neighboring tumor cells.

It has previously been shown that adenovirus 11p exhibits high affinity to hematopoietic cells (Segerman et al. 2000. Adenovirus types 11p and 35 p show high binding efficiencies for committed hematopoietic cell lines and are infective to these cell lines, J Virol 74(3), 1457-1467). This article however gives insufficient information on the interaction between the virus and normal cells or stem cells. Importantly, the experimental set-up results in the information being specific for cancerous cells, and the relevance on non-cancerous or pre-cancerous cells can be disputed.

U.S. Pat. No. 7,459,153 and the corresponding EP 1 348 030 disclose the complete nucleic acid sequence of native adenovirus type 11. The contents of U.S. Pat. No. 7,459,153 is hereby incorporated by reference. The sequence is also available through the EBI/EMBL database under accession No. AF532578.

Oncolytic effects of a recombinant adenovirus vector have been described (Sandberg et al. 2009. Replication-competent Ad11p vector (RCAd11p) efficiently transduces and replicates in hormone-refractory metastatic prostate cancer cells. Hum Gene Therapy 20, 361-373). However, this publication does not provide any information as to how this vector was constructed.

In view of the above, it remains a problem to make available an adenoviral vector with both high infectivity to important cell types, a low prevalence in society, and which allows the insertion of heterologous genes while maintaining replication competence.

Further problems and their solutions will be evident from the description and examples, as read by a person skilled in the art.

### BRIEF DESCRIPTION OF THE INVENTION

The present inventors have found that it is possible to insert heterologous nucleic acid fragments into the genome of the adenovirus Ad11p thereby providing recombinant adenovirus vectors which unexpectedly maintaining replication competence and infectivity.

Accordingly, in one aspect the present invention provides an isolated nucleic acid comprising a sequence having at least 90%, preferably at least 95%, more preferably at least 98%, most preferably at least 99% sequence identity to the sequence set forth in SEQ ID NO:1, further comprising one or more inserts of a heterologous nucleic acid fragment.

In one embodiment said insert is a heterologous gene of interest.

In another embodiment said insert encodes a siRNA, shRNA, or a micro RNA.

Preferably said heterologous nucleic acid fragment has a size of between 100 and 5,000 base pairs, such as a between 500 to 2,000 base pairs.

Preferably said heterologous nucleic acid fragment is cloned into the E1 region of the Ad11p genome and/or into the E3 region of the Ad11p genome.

By the E1 region is meant the E1A and E1B regions. The E1A, E1B and E3 regions are defined in FIG. 7.

In one preferred embodiment one heterologous nucleic acid fragment is cloned into the E1 region, and a second heterologous nucleic acid fragment is cloned into the E3 region.

Preferably said heterologous nucleic acid fragment is cloned into the E1 region at a position corresponding to between nucleotide 247 and nucleotide 568, more preferably between nucleotide 382 and nucleotide 568, even more preferably between nucleotide 382 and nucleotide 479, even more preferably between nucleotide 436 and nucleotide 479 of SEQ ID NO:1.

Most preferably said heterologous nucleic acid fragment is cloned into the E1 region at a position corresponding to nucleotide 247, 382, 436, 479, 451, 479, or 568 of SEQ ID NO:1

Preferably said heterologous nucleic acid fragment is cloned into the E3 region at a position corresponding to between nucleotide 26864 and nucleotide 30633, more preferably between nucleotide 27836 and nucleotide 29526, or even more preferably between nucleotide 27836 and nucleotide 28356, between nucleotide 28356 and nucleotide 29481, between nucleotides 28901 and nucleotide 28919, or between nucleotide 28920 and nucleotide 29526, of SEQ ID NO:1.

Most preferably said heterologous nucleic acid fragment is cloned into the E3 region at a position corresponding to nucleotide 27836 or nucleotide 28356 of SEQ ID NO:1.

In one embodiment part of the E3 region is replaced by the insertion of said heterologous nucleic acid fragment.

In one embodiment the expression of said heterologous nucleic acid fragment is under the control of a heterologous enhancer and/or promoter.

Preferably said heterologous enhancer and/or promoter is selected from heterologous viral promoters, and mammalian tissue specific promoters.

By mammalian tissue specific promoter is meant a promoter mediating the expression of the heterologous nucleic acid fragment mainly specifically in one or more mammalian tissue.

In another embodiment the expression of said heterologous nucleic acid fragment is under the control of adenoviral endogenous gene expression machinery or an adenoviral endogenous enhancer and/or promoter.

In one embodiment said heterologous nucleic acid fragment encodes a protein selected from the group consisting of viral proteins, antigenic determinants of a pathogenic organism, tumour-specific antigens, human therapeutic proteins, and cytokines

Preferably said heterologous nucleic acid fragment encodes a viral protein selected from the group consisting of ADP, E1A, and p300.

Preferably said heterologous nucleic acid fragment encodes a human protein selected from the group consisting of: Rb, CFTR, p16, p21, p2′7, p57, p′73, C-CAM, APC, CTS-1, zacl, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, fus, interferon α, interferon β, interferon γ, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, RaplA, cytosine deaminase, Fab, ScFv, BRCA2, zacl, ATM, HIC-1, DPC-4, FHIT, PTEN, ING1, NOEY1, NOEY2, OVCA1, MADR2, 53BP2, IRF-1, Rb, zacl, DBCCR-1, rks-3, COX-1, TFPI, PGS, Dp, E2F, ras, nzyc, neu, raf, erb, fins, trk, ret, gsp, hst, abl, VEGF, FGF, PEDF, thrombospondin, BAI-1, GDAIF, and MCC.

In another embodiment said heterologous nucleic acid fragment encodes a reporter protein selected from the group consisting of: fluorescent proteins, luminescent proteins, and enzymes.

Preferably said reporter protein is selected from the group consisting of green fluorescent protein, enhanced green fluorescent protein, yellow fluorescent protein, blue fluorescent protein, cyan fluorescent protein, red fluorescent protein, luciferase, β-galactosidase, chloramphenicol acetyltransferase.

In another aspect the present invention provides a recombinant replication competent adenovirus vector based, comprising a nucleic acid according to the invention.

Accordingly one important aspect of the present invention is a recombinant replication competent adenovirus vector comprising a sequence having at least 90%, preferably at least 95%, more preferably at least 98%, most preferably at least 99% sequence identity to the sequence set forth in SEQ ID NO:1, further comprising one or more inserts of a heterologous nucleic acid fragment, wherein said heterologous nucleic acid fragment is cloned into the E1 region of the Ad11p genome and/or into the E3 region of the Ad11p genome.

By the E1 region is meant the E1A and E1B regions. The E1A, E1B and E3 regions are defined in FIG. 7.

Preferably said heterologous nucleic acid fragment is cloned into the E1 region at a position corresponding to between nucleotide 247 and nucleotide 568, more preferably between nucleotide 382 and nucleotide 568, even more preferably between nucleotide 382 and nucleotide 479, even more preferably between nucleotide 436 and nucleotide 479 of SEQ ID NO:1.

Most preferably said heterologous nucleic acid fragment is cloned into the E1 region at a position corresponding to nucleotide 247, 382, 436, 479, 451, 479, or 568 of SEQ ID NO:1

Preferably said heterologous nucleic acid fragment is cloned into the E3 region at a position corresponding to between nucleotide 26864 and nucleotide 30633, more preferably between nucleotide 27836 and nucleotide 29526, or even more preferably between nucleotide 27836 and nucleotide 28356, between nucleotide 28356 and nucleotide 29481, between nucleotides 28901 and nucleotide 28919, or between nucleotide 28920 and nucleotide 29526, of SEQ ID NO:1.

Most preferably said heterologous nucleic acid fragment is cloned into the E3 region at a position corresponding to nucleotide 27836 or nucleotide 28356 of SEQ ID NO:1.

In yet another aspect the present invention provides pharmaceutical compositions comprising a recombinant replication competent adenovirus virus according to the invention, optionally in combination with a suitable excipient.

In yet another aspect the present invention provides recombinant replication competent adenovirus vector according to the invention for use as a medicament.

In yet another aspect the present invention provides use of a recombinant replication competent adenovirus vector according to the invention in the manufacture of a medicament for the therapeutic, prophylactic or diagnostic treatment of a disease.

Preferably said disease is selected from the group consisting of a viral infectious diseases, bacterial infectious diseases, cancer diseases, vascular diseases, cardiovascular diseases, immunological diseases, liver diseases, and parasitic diseases.

Preferably said viral infectious disease is selected from the group consisting of viral diseases caused by HCV, HBV, HIV, and influenza A virus.

Preferably said bacterial infectious disease is selected from the group consisting of tuberculosis, and septic chock.

Preferably said parasitic disease is malaria.

Preferably said cancer disease is selected from the group consisting of: lung cancer, breast cancer, ovarian cancer, prostate cancer, colorectal cancer, bladder cancer, kidney cancer, pancreatic cancer, haematological malignancies, liver cancer, tumours of neural origin, head and neck cancer, and osteosarcoma.

In yet another aspect the present invention provides methods of treating a mammalian patient having a cancer disease, said method comprising administering to the patient the pharmaceutical composition according to the invention.

According to one embodiment the pharmaceutical composition is administered locally to a tumour site.

According to another embodiment the pharmaceutical composition is administered intravesically or intraductally.

Intravesical administration can be a therapeutic advantage e.g. in the treatment of bladder cancer. Intraductal administration can be a therapeutic advantage e.g. in the treatment of breast cancer.

According to yet another embodiment the pharmaceutical composition is administered by intravenous injection.

According to one embodiment the method further comprises administering a chemotherapeutic agent to the patient.

According to another embodiment the method further comprises treating the patient with radiation therapy.

In yet another aspect the present invention provides use of a recombinant replication competent adenovirus according to the invention for gene therapy.

In yet another aspect the present invention provides a mammalian cell comprising a recombinant replication competent adenovirus according to the invention. Transfected mammalian cells can be used for the production of the vectors according to the invention, and for the production of recombinant proteins.

In yet another embodiment the present invention provides methods of expressing a protein or gene product or an expression product which comprises infecting or transfecting a cell in vitro with a recombinant replication competent adenovirus according to the present invention and optionally extracting, purifying or isolating the protein, gene product or expression product from the cell.

In yet another embodiment the present invention provides use of a recombinant replication competent adenovirus vector according to the present invention for the identification of anti-viral compounds.

In yet another embodiment the present invention provides methods for the identification of anti-viral compounds comprising the use of a recombinant replication competent adenovirus vector according to the present invention.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The present invention will be described in further detail below, in the description and attached sequence listing and drawings, in which

FIG. 1. A. Flow cytometric analysis of expression of human CD46, CAR, αᵥβ₃ integrin, and αᵥβ₅ integrin on the surface of metastatic cells derived from human prostate (PC-3, DU145, and LNCaP) and control cell lines (A549 and HEK293). B. Graph of expression of different cell-surface molecules (CD46, CAR, αᵥβ₃, αᵥβ₅, CD46, CD80, and CD86) in the prostate cancer cell lines PC-3, DU145, and LNCaP, and in the control cell lines A549 and HEK293. Significant difference to PC-3 cells is indicated on top of each bar, *P<0.05; **P<0.01; *** P<0.001.

FIG. 2. Transduction analysis of prostate carcinoma cell lines (DU145, PC-3, and LNCaP) and control cell lines (A549 and HEK293). The cells were infected with RCAd11pGFP at 0.01, 0.05, 0.1, 0.5, and 1 pg virus particles per cell. The transduction efficiency was assessed by flow cytometry of GFP expression at 48 h postinfection, respectively. Significant difference to 0.01 pg virus per cell is indicated on top of each bar, *P<0.05; **P<0.01; ***P<0.001.

FIG. 3. Transduction was reduced by antibodies to CD46, by Ad11prfib, and integrins αᵥβ₃ and αᵥβ₅. A. Monoclonal anti-CD46 antibody. B. Polyclonal anti-CD46 antibody. C. Graph of blockage by Ad11prfib at different concentrations (μg/10⁶ cells). Positive controls were infected with RCAd11pGFP vector at concentrations of 0.5 pg per cell for A549 and 1.0 pg per cell for 293, DU145, PC-3 and LNCaP. D. Anti-Ad11prfib antiserum. E. Monoclonal αᵥβ₃ antibody and αᵥβ₅antibody. Significant difference to control is indicated on top of each bar, *P<0.05; **P<0.01; ***P<0.001.

FIG. 4. A. The extent of RCAd11p-induced cytopathic effect (CPE) did not correlate with p53 genotype. RCAd11p-induced cytolysis was detected in the p53-positive A549 and HEK293 lines, and in the p53-negative PC-3 line and the p53 mutant DU145 and LNCaP cell lines. Infection was done at 0.1 pg/cell and 1 pg/cell as described in Materials and Methods. Dose-dependent induction cell CPE was evaluated by microscopy at 48 h p.i. B. RCAd11p viral DNA replication was assessed in p53⁺ and p53⁻ cell lines. Prostate carcinoma cells (PC-3, DU145, and LNCaP) and control cells (A549 and HEK293) were mock-infected and infected with RCAd11pGFP at 0.1 pg/cell (multiplicity of infection of 5) as described in Materials and Methods. Viral DNA was harvested from infected cells at the indicated times postinfection (p.i.) DNA isolated from an equal number of cells (2×10⁵) was digested with the restriction endonuclease BamHI. C. The viral DNA was detected using a NanoDrop spectrophotometer.

FIG. 5. A: Comparison of the oncolytic effect of RCAd11pGFP and Ad11pwt. A549, 293, PC-3, DU145, and LNCaP cells were infected with 10-fold diluted viruses, starting at 1 pg/cell in all tests. The cells were stained by crystal violet 6 days post infection. The RCAd11pGFP virus was slightly more oncolytic than wild-type Ad11. B. Cell viability was measured by XTT assay 6 day post infection. The results from XTT assays were presented as absorbance at 490 nm. The graph represents the average of four replicate samples. C. One-step grow curve indicated the replication capacity of the virus in the three prostate cancer cell lines.

FIG. 6. In vivo oncolytic model with RCAd11p vector. 10⁷ PC-3 cells /tumor were subcutaneously transplanted into left and right frank region of Balb/c nude mice, the control group mice were injected with only PBS. As PC-3 tumor grew at least 75 mm3 after 3 week injection, 50 μg of RCAd11p/tumor was injected into mice with intratumor administration. A. tumor volume with or mock treated by RCAd11p was recorded weekly. The mice were scarified approximately 6 weeks after viral injection. B. statistics of the tumour growths 3 and 4 weeks post infection.

FIG. 7. Genome organization of Ad11. The linear double-stranded genome is depicted in the centre as a double line, with the inverted terminal repeats (ITRs) at each end. Transcription units are shown as arrows, relative to their position and orientation in the Ad11 genome. These include early genes (E1A, E1B, E2A, E2B, E3 and E4), genes expressed at intermediate times of infection (1× and IVa2) and late genes (L1-L6). All of the late genes are expressed from the major late promoter (MLP) and contain the tripartite leader (TPL) at their 5′-ends. A triangle represents the virus-associated (VA) RNA. One map unit (m.u.) is equivalent to 347.94 bp.

FIG. 8. Construction of the RCAd11pGFP vector.

For the construction of replication competent adenovirus vectors, three alternative insertion sites ain the E1 region of the genome were identified. A foreign gene can be inserted at the beginning of adenovirus genome between nucleotide 1 and nucleotide 568. However, the insertion at the location between nucleotide 247 and nucleotide 568 would produce a competent vector of better quality. Furthermore, insertion of the foreign gene between nucleotides 382 and nucleotide 568 of the adenovirus genome will result in an even better infectious vector. The insertion of the foreign gene between nucleotide 436 and nucleotide 479 or even more preferably at the site corresponding to nucleotide 451 can consequently yield best infectious adenovirus vectors that have a replication competence comparable to their wild type counterparts.

FIG. 9. Examples RCAd11p vectors with insertions in the E3 region.

To generate replication competent adenovirus vector in the E3 region without negative effect on the replication capacity, the entire e3 region between nucleotide 26864 and nucleotide 30633 bp or the region between nucleotide 26864 and nucleotide 29525 can be replaced with one open reading frame (ORF) or one expression cassette. Alternatively, insertion of an ORF between nucleotide 28901 and nucleotide 28919 generates infectious adenovirus vectors. Furthermore, the ORF between nucleotide 27836 and nucleotide 28356 or between nucleotide 28356 and nucleotide 29482 can be replaced by one new ORF or one expression cassette so that the new constructs can function as wild type virus. All described constructs consequently produce replication-competent adenovirus vectors without effect on replication capacity.

FIG. 10. In vivo oncolytic model with RCAd11p vector. T84 cells derived from a lung metastasis of a colorectal adenocarcinoma and HT 29 derived from colorectal adenocarcinoma. 10⁷ T84 cells /tumor or H-29 cells in 0.2 ml were subcutaneously transplanted into left and right flank region of Balb/c nude mice, the control group mice were injected with only PBS. As colon tumor grew up to at least 75 mm³ 3 weeks after injection, 50 μg of RCAd11p/tumor was injected into mice with intratumoral administration. Tumor volume was recorded weekly. The mice were sacrificed approximately 6 weeks after viral injection. (o) control, (▪) mice treated with RCAd11pGFP, (▴) mice treated with wild-type Ad11p.

### DETAILED DESCRIPTION OF THE INVENTION

The term “substantially homologous” as used herein refers to the ability of two nucleic acids to hybridise under at least moderately stringent hybridisation conditions. Stringency of hybridisation is a term of the art that refers to the conditions used for a hybridisation reaction whereby complementary single strands of nucleic acid join to one another to form double-stranded nucleic acid with some degree of mismatch, the degree of which is a function of the stringency-used. In particular, the stringency will depend upon the size and composition of the strands of nucleic acid that are caused to react, the degree of mismatching allowed, the desired cross reactivity, and the like. The degree of stringency can be affected by ionic conditions employed and temperature, among others, as is well known in the art (e.g. Sambrook et al., Molecular cloning: A laboratory manual, second edition, 1989).

The terms “functionally homologous” and “functionally similar” refers to homologies and similarities accounting for the same function or behaviour with respect to tropism, affinity to specific cells and immune response inducing behaviour.

The present inventive adenoviral vector preferably further comprises heterologous nucleic acid fragments, which will typically encode, and express within a host cell, a product that has therapeutic and/or prophylactic utility. The term “heterologous nucleic acid fragment” is used herein to refer to any sequence of DNA or RNA, in particular DNA, functionally inserted into a vector according to the present invention that is foreign to the adenoviral genome. Such heterologous nucleic acid fragment may constitute a gene, a portion of a gene, or any other nucleic acid sequence, including but not limited to a sequence that encodes RNA, anti-sense RNA, a synthetic oligonucleotide, and/or a polypeptide. Heterologous nucleic acid fragment having therapeutic utility include genes that encode a missing or impaired gene function, and genes influencing the behaviour of the cell, such as so called suicidal genes. Foreign nucleic acids having prophylactic utility include genes that encode a gene product that has an ability to prevent disease directly or indirectly, e.g. by providing a source of a polypeptide or other antigen to elicit an immune response thereto.

The term “replication competent” as used herein refers to the ability of a vector to replicate in the host cell.

The term “therapeutic and/or prophylactic agent” and the term “product having therapeutic and/or prophylactic utility” are used as equivalents and are meant to comprise inter alia antigens and immunostimulating agents, such as cytokines etc.

The present invention further provides the purified and isolated DNA of an adenovirus having the nucleotide sequence of SEQ ID NO: 1 or a sequence hybridising thereto under stringent conditions or substantially homologous therewith, further comprising an inserted heterologous nucleic acid fragment.

The present invention provides a novel adenoviral vector comprising a sequence with at least 90% such as at least 95% identity to the sequence of SEQ ID NO: 1, in particular an adenoviral vector comprising a sequence with at least 98% identity such as at least 99% identity to the sequence of SEQ ID NO: 1 or most preferably an adenoviral vector comprising a sequence substantially homologous to the sequence of SEQ ID NO: 1, further comprising an inserted heterologous nucleic acid fragment.

To determine the percent identity of two nucleic acids, the sequences are aligned for optimal comparison purposes (e.g., gaps can be introduced in the sequence of a first nucleic acid sequence for optimal alignment with a second amino or nucleic acid sequence). The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences (i.e., percent identity=number of identical positions/total number of positions (e.g., overlapping positions)×100). In one embodiment, the two sequences are the same length.

To determine percent sequence identity, a target nucleic acid sequence is compared to the identified nucleic acid sequence using the BLAST 2 Sequences (B12seq) program from the stand-alone version of BLASTZ containing BLASTN version 2.0.14. This stand-alone version of BLASTZ can be obtained from the U.S. government's National Center for Biotechnology Information web site (world wide web at ncbi.nlm.nih.gov). Instructions explaining how to use the B12seq program can be found in the readme file accompanying BLASTZ.

B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seql.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. The following command will generate an output file containing a comparison between two sequences: C:\B12seq -i c:\seq1.txt -j c:\seq2.txt -p blastn -o c:\output.txt -q -1-r2. If the target sequence shares homology with any portion of the identified sequence, then the designated output file will present those regions of homology as aligned sequences. If the target sequence does not share homology with any portion of the identified sequence, then the designated output file will not present aligned sequences.

Once aligned, a length is determined by counting the number of consecutive nucleotides from the target sequence presented in alignment with sequence from the identified sequence starting with any matched position and ending with any other matched position. A matched position is any position where an identical nucleotide is presented in both the target and identified sequence. Gaps presented in the target sequence are not counted since gaps are not nucleotides. Likewise, gaps presented in the identified sequence are not counted since target sequence nucleotides are counted, not nucleotides from the identified sequence.

The percent identity is determined by dividing the number of matches by the length of the sequence set forth in an identified sequence followed by multiplying the resulting value by 100. For example, if a sequence is compared to the sequence set forth in SEQ ID NO:1 (the length of the sequence set forth in SEQ ID NO:1 is 34 794) and the number of matches is 33 401, then the sequence has a percent identity of 96 (i.e., 33 401÷34 794*100=96) to the sequence set forth in SEQ ID NO:1.

The present invention also provides a purified and isolated DNA sequence comprising the nucleotide sequences defined by positions 30812-31789 and 18255-21101 in SEQ ID NO:1, further comprising an inserted heterologous nucleic acid fragment.

In particular, the present invention provides a novel adenoviral vector comprising the sequence of SEQ ID NO: 1 or a fragment thereof, further comprising one or more inserts of a heterologous nucleic acid fragment.

Further, the invention provides a vector as above, wherein said vector further comprises a foreign nucleic acid fragment that can express in a human a therapeutic and/or prophylactic agent or a foreign nucleic acid which in itself has a therapeutic and/or prophylactic utility.

The present invention further provides a method of gene therapy comprising the administration to a human, preferably a human patient, in need of such gene therapy a therapeutically effective amount of a vector as defined above.

The present invention further provides the use of a vector comprising a sequence of SEQ ID NO: 1 or a sequence substantially homologous therewith, further comprising one or more inserts of a heterologous nucleic acid fragment in gene therapy.

The present invention further provides the use of a vector comprising a sequence of SEQ ID NO: 1 or a sequence substantially homologous therewith, in gene therapy in cancer therapy. The invention also encompasses the use of a vector comprising a sequence of SEQ ID NO: 1 or a sequence substantially homologous therewith, in gene therapy in vascular therapy.

The present invention further provides use of a vector comprising a sequence of SEQ ID NO: 1 and an inserted selected therapeutic gene operatively linked to regulatory sequences which direct expression of said gene in the production of a medicament for treating a patient having an acquired or inherited genetic defect.

The invention makes available the use of a vector as defined above for the transfection of human cells chosen among cells of neural origin, and in particular for the infection of human cells chosen among glioblastoma, neuroblastoma and medulloblastoma, as wells haemopoietic progenitor cells.

The invention makes available the use of a vector as defined above for the transfection of human cells chosen among hepatoma cells, breast cancer cells, prostatic cancer cells and endothelial cells.

The invention makes available the use of a vector as defined above for the transfection of human cells chosen among dendritic cells.

An adenoviral vector according to the present invention bearing a heterologous nucleic acid fragment encoding a product that has therapeutic and/or prophylactic utility may be administered to a human or other human patient, preferably suspended in a biologically compatible solution or a pharmaceutically acceptable delivery vehicle. A suitable vehicle is sterile saline solution. Other aqueous and non-aqueous isotonic sterile injection solutions and aqueous and non-aqueous sterile suspensions known to be pharmaceutically acceptable carriers and well known to those of skill in the art may be employed for this purpose.

A composition for administration of an adenoviral vector according to the invention may be formulated to contain other components, such as adjuvants, stabilisers, pH adjusters, preservatives and the like. Such components are well known to persons skilled in the relevant art of viral gene therapy.

The adenovirus vectors according to the invention are administered in a pharmaceutically effective amount, i.e. an amount that is effective to transfect the desired cells—in the chosen route of administration—and provide sufficient level of expression of the selected gene to provide a therapeutic benefit.

Conventional and pharmaceutically acceptable routes of administration include, but are not limited to, intravesicular, intraductal, intranasal, intramuscular, intratracheal, subcutaneous, intradermal, rectal, oral, instillation into the urinary bladder and other parenteral routes of administration. Routes of administration may be combined, if desired, or adjusted depending upon the therapeutic goal, e.g. elicitation of immunity, and primarily on the nature of the disease being treated.

The adenovirus vectors according to the invention can be propagated and produced in any suitable cell line, such as in Vero cells from monkey kidney, A-549 cells, human embryonic retinoblast (HER) cells, such as HER 911, human embryonic kidney (HEK) cells, such as HEK 293, as well as other cell lines approved for human vaccine production such as the MRC-5, the WI-38 and the FRhL-2 cell lines.

Accordingly, the present invention further provides cells transfected with adenovirus vectors according to the invention, and methods for the production of adenovirus vectors according to the invention using such cells.

### EXAMPLES

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Replication-Competent Ad11p Vector (RCAd11p) Efficiently Transduces and Replicates in Hormone-Refractory Metastatic Prostate Cancer Cells

### Materials and Methods

### Cell Lines and Culture Conditions

The human embryonic kidney cell line 293 (HEK-293), which expresses the adenoviral E1A and E1B gene products, was purchased from Microbix Biosystems (Toronto, ON, Canada). The tumor cell lines LNCaP (from prostate; metastatic site: left supraclavicular lymph node carcinoma), DU 145 (from prostate; metastatic site: brain carcinoma), and PC-3 (from prostate; metastatic site: bone adenocarcinoma) were obtained from the American Type Culture Collection (ATCC, Manassas, Va.). The cell lines were all cultured at 37° C. in 5% CO2, using culture medium recommended by the ATCC. The HEK-293 cell line and human A549 cell line (respiratory oat cell carcinoma) were grown at 37° C. in Dulbecco's modified Eagle's medium (Sigma-Aldrich, St. Louis, Mo.), 20 mM HEPES (pH 7.4), penicillin-streptomycin (100 IU/ml and 100 mg/ml, respectively), and with 10% fetal bovine serum (FBS) for 293 cells but only 5% for A549 cells. On viral infection, the FBS concentration was reduced to 5% for 293 cells and 2% for A549 cells. Adenoviruses and vectors Ad11 prototype (strain Slobitski) was propagated in A549 cells and purified on CsCl as described elsewhere (Mei et al., 1998). The virion band was collected. The buoyant density was checked with a refractometer and the viral concentration was measured in a spectrophotometer (Saveen & Werner, Limhamn, Sweden). The antiserum against purified virions of adenovirus type 11 was as previously described by Wadell et al. (1999. “Adenoviruses.” Manual of Clinical Microbiology. ASM Press Washington, D.C.).

### RCAd11p Vector

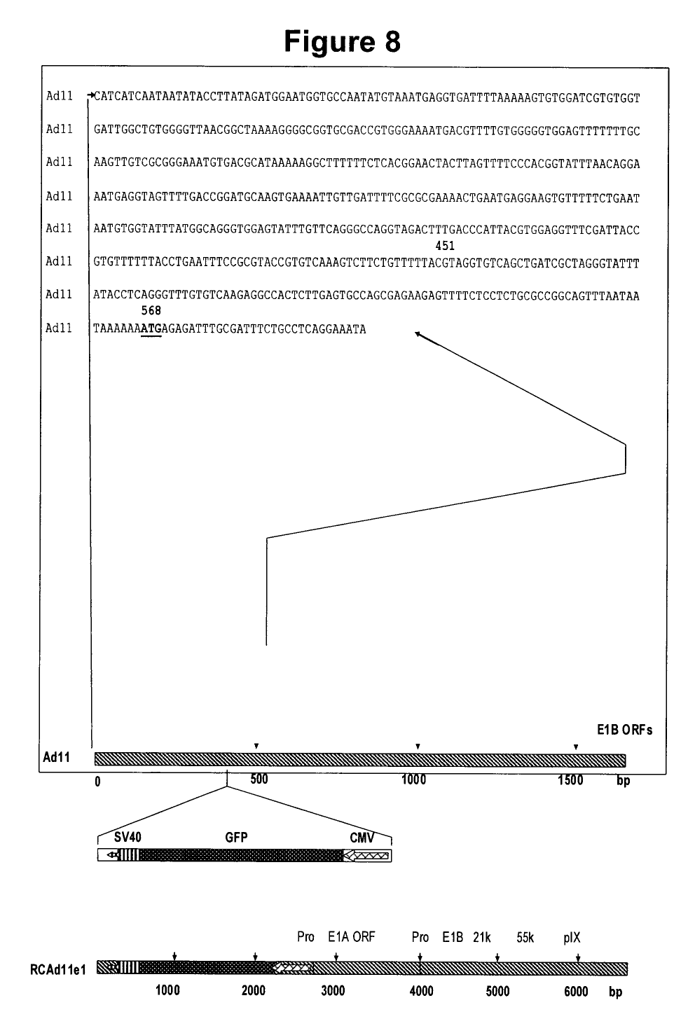

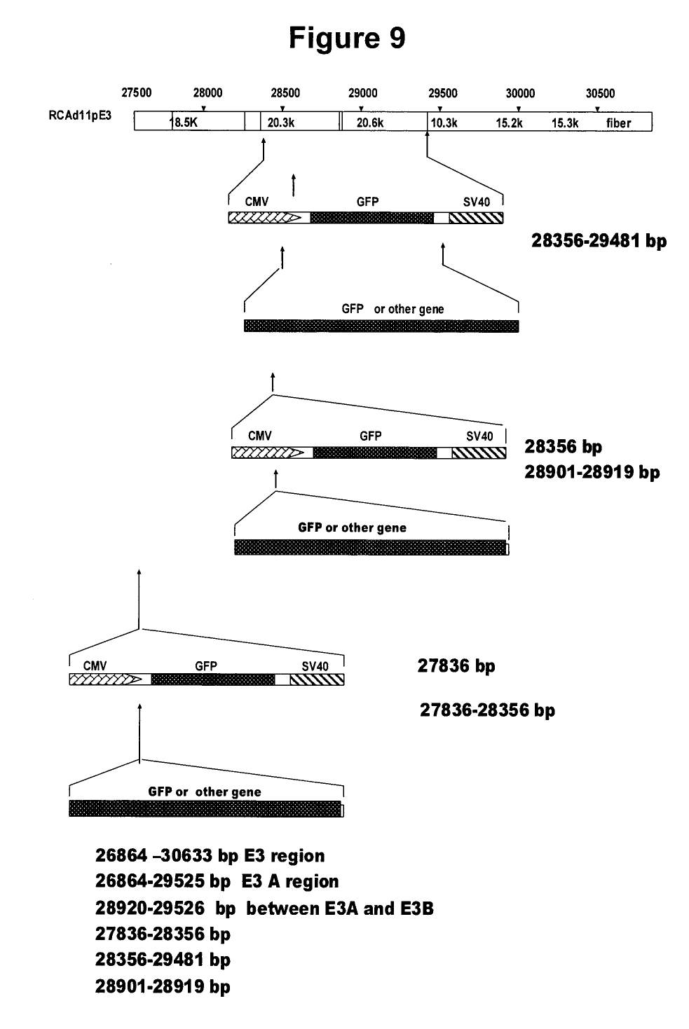

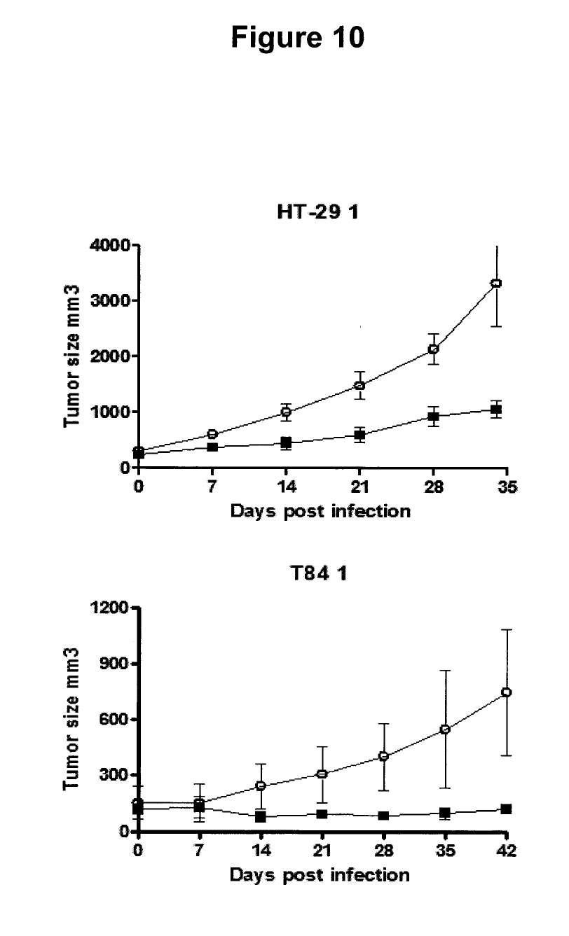

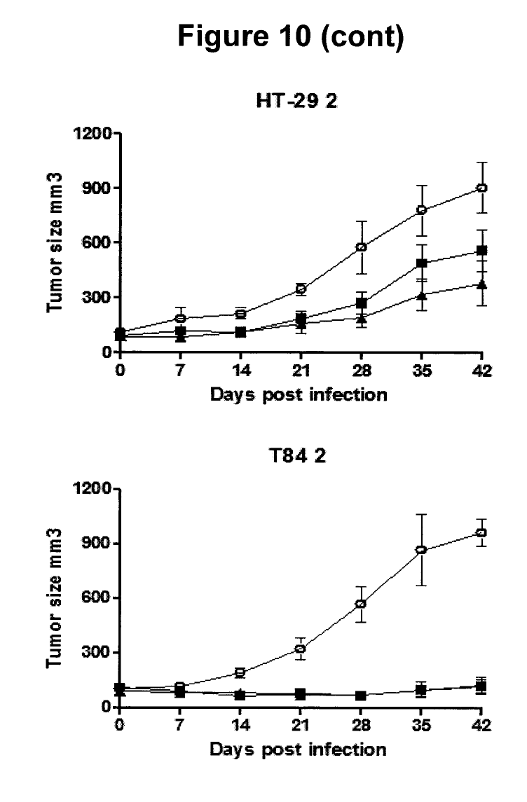

The replication-competent RCAd11p vector was constructed by insertion of an expression cassette comprising the CMV promoter, the GFP gene, and the SV40 poly(A) signal into the Ad11p genome. The insertion was made in the E1 region, at a position corresponding to nucleotide 451 in SEQ ID NO:1 Construction ofg RCAd11pGFP is described in FIG. 8.

### Determination of Cell Surface Molecules by Flow Cytometry

Cytometric analysis was used to determine the levels of various molecules on the cell surface. Briefly, the expression levels of CAR, CD46, CD80, CD86, αᵥβ₃, and αᵥβ₅ on the cells were investigated with mouse monoclonal antibodies: anti-CAR (clone RmcB; Chemicon International/Millipore, Temecula, Calif.) diluted 1:200, mouse anti-human CD46 monoclonal antibody (169-1-E4.3; Ancell, Bayport, Minn.) or rabbit antihuman CD46 polyclonal antibody (provided by J.P. Atkinson, Washington University School of Medicine, St. Louis, Mo.) diluted 1:200, anti-human CD80 (B7-1; Ancell) diluted 1:50, anti-human CD86 (B7-2; Ancell) diluted 1:200, anti-human integrin αᵥβ₃, (MAB1976; Chemicon International/Millipore) diluted 1:200, or anti-human integrin αᵥβ₅ (MAB1961, Chemicon International/Millipore) diluted 1:1000. Fluorescence staining of membranes was performed on a single-cell suspension by adding one of the previously mentioned monoclonal antibodies followed by fluorescein isothiocyanate (FITC)-conjugated secondary antibodies (FITC-conjugated goat anti-mouse Fab F2653; Sigma-Aldrich). Flow cytometry (FACScan; BD Biosciences, San Jose, Calif.) was performed at 488 nm, using LYSYS II software (BD Biosciences), by gating the distribution of the negative control sample for each individual cell line. This setting was used to determine the geometric mean value and percentage of cells expressing each of the previously mentioned markers for each individual cell line.

### Detection of Virus-Mediated Gene Delivery

A standard number of cells of each cell line (2×10⁵ cells) was used to seed each well of a 24-well dish. On the next day, the cells were transduced with 0.01, 0.05, 0.1, 0.5, or 1 pg of RCAd11pGFP vector per cell for 1 hr. Forty-eight hours later, the cells were harvested and centrifuged at 300×g for 5 min. They were then fixed with 3 ml of 2% paraformaldehyde (PFA) for 15 min at room temperature, centrifuged at 300×g for 5 min, washed with 3 ml of 2% FBS in phosphate-buffered saline (PBS), centrifuged at 300×g, and then resuspended in 300 ml of 2% FBS in PBS (bovine serum albumin [BSA]-PBS). Finally, green fluorescent protein (GFP) expression was measured by flow cytometry. The amount of viral particles (VP) is represented by the measurement of picograms, with 1 pg representing approximately 3600 physical particles and corresponding to 50 times the 50% tissue culture infective dose (TCID₅₀), because when RCAd11pGFP vector was titered it was demonstrated that 1 TCID₅₀ unit corresponds to 72 VP. Virion concentration was determined by spectrophotometry at 260 and 330 nm according to the following formula: 1 unit of absorbance at A260-A330 is equivalent to 10¹² particles/ml or 280 mg of viral particles per milliliter.

Antibody Blocking of RCAd11pGFP-Mediated Transduction

The cells were grown in 12-well plates overnight and incubated with either monoclonal anti-CD46 antibody or rabbit polyclonal anti-CD46 antibody at a dilution of 1:200 in PBS for 30 min at 37° C. They were infected with virus corresponding to 0.5 pg/cell (for A549 cells) or 1 pg/cell (for 293, PC-3, DU 145, and LNCaP cells) at 37° C. for 1 hr. The cells were then washed twice with BSA-PBS, and cultivated at 37° C. for 48 hr. The infected cells were detached with EDTA and 0.1% trypsin in PBS and resuspended in medium with 10% FBS. They were centrifuged and resuspended at 4° C. for 1 hr in 1 ml of PBS containing 2% PFA. The cells were centrifuged again and the cell pellets were resuspended in 200 ml of PBS and placed in the wells of a 96-well plate, which was kept at 4° C. for 30 min. The cells were then washed with BSA-PBS and transferred in the same buffer to flow cytometry tubes.

Assessment by Flow Cytometry of Capacity of Ad11prfib and Anti-Ad11prfib Antiserum to Block GFP Expression

Various amounts (0.5, 3, 6, and 10 μg) of Ad11p recombinant fiber knob protein (Ad11prfib) were added to 10⁶ cells and incubated on ice for 30 min. The cells were then inoculated with RCAd11pGFP vector at 0.5 pg/cell (for A549 cells) or 1.0 pg/cell (for 293, DU 145, PC-3, and LNCaP cells) and incubated at 37° C. for another 30 min. They were washed once with PBS containing 2% FBS, and then fresh culture medium was added. The number of infected cells was assessed by flow cytometry at 48 hr postinfection. The cells were seeded in 12-well plates and grown overnight. Serially diluted anti-Ad11p recombinant fiber (rfib) antiserum was premixed with RCAd11pGFP virions at 2 pg/cell for 1 hr at room temperature, and then moved to 12-well plates. Other steps were as described previously.

### Isolation of Viral DNA

One million cells of each cell line were inoculated with RCAd11pGFP (0.1 pg/cell) and harvested at 24, 48, and 72 hr postinfection. Viral DNA was purified according to the method of Shinagawa and colleagues (1983). The amount of purified DNA was measured with a NanoDrop (Saveen & Werner). The DNA was digested with BamHI restriction enzyme and separated on a 1% agarose gel.

### Viral Protein Western Blot Analysis

The five cell lines were seeded at 1.5×10⁵ cells per well and infected with RCAd11pGFP or wild-type Ad11p (Ad11pwt) at 0.1 pg/cell. At 96 hr postinfection, the cells were collected by centrifugation at 800 rpm for 5 min. The cell pellets were washed once with PBS and boiled in loading buffer. Samples of 1×10⁵ cells were loaded and separated on NuPage 12% Bis-Tris gels (Invitrogen, Carlsbad, Calif.) and then the gels were transferred to nitrocellulose. The viral proteins were detected with virion antibody from rabbit and horseradish peroxidase (HRP)-conjugated anti-rabbit (Dako, Glostrup, Denmark). Cytotoxicity assay To stain the cells with crystal violet, the medium was removed and the cells were fixed for 3 min in 3.7% PFA at room temperature. They were then washed with PBS and incubated for 3 min in 1% crystal violet in 70% ethanol. After staining, the cells were rinsed three times with water and air dried for photography.

### Cell Proliferation Assay

To evaluate the oncolytic effect of RCAd11pGFP in prostate cancer cells, cells were infected with RCAd11pGFP and Ad11pwt at 10⁻⁴, 10⁻³, 10⁻², 10⁻¹, and 10⁰ viral pg per cell, corresponding to 0.36, 3.6, 36, 360, and 3600 VP/cell. Cell viability was determined by XTT (sodium 3-[1-(phenylaminocarbonyl)-3,4-tetrazolium]-bis(4-methoxy-6-nitro)benzene sulfonic acid hydrate) assay at 6 days postinfection (cell proliferation kit II; Roche Applied Science, Indianapolis, Ind.). Quantitation was performed colorimetrically at a wavelength of 490 nm.

One-Step Growth Curves of Ad11pwt and RCAd11pGFP

Monolayers of DU145, PC-3, and LNCaP cells were infected with either RCAd11pGFP or wild-type Ad11 (Ad11pwt) at 0.2 pg/cell. At 24, 48, 72, and 96 hr postinfection, duplicate cell samples were harvested and lysed by three cycles of freeze-thawing, and the virus in the supernatants was assayed in HEK-293 cell monolayers. One-step growth curves of the viruses were performed in LNCaP cells with methyltrienolone (R1881) in the medium. Viral yields were titrated 96 hr after infection. Duplicate sets of cells were infected, and the assays were carried out four times. A classical TCID₅₀ assay was used to measure the viral titration.

### Animal Experiments

BALB/c nude mice were obtained from Taconic (Ry, Denmark) at 3-4 weeks of age and they were quarantined for at least 1 week before study. PC-3 cells were grown in F12K medium with 10% FBS, 2 mM 1-glutamine, and penicillin and streptomycin, until the cells were confluent. They were harvested by two consecutive trypsinizations, centrifuged at 300×g for 5 min, washed twice, and resuspended in PBS, and the cell density was adjusted to 5×10⁷ cells/ml. Then 1×10⁷ cells in 0.2 ml were injected subcutaneously into each nude mouse. When tumors with a volume of 75 mm³ developed animals were randomly assigned to three groups: two treated groups received one intratumoral injection of 50 mg of RCAd11p vector or Ad11pwt virus that was diluted in a volume of 100 ml of PBS; one untreated group received PBS only. Tumor axes were measured weekly with vernier calipers, and tumor volume was calculated according to the following simplified formula: (l×w×d)/2.

### Statistical Analysis

Statistical analyses (t tests) were performed with GraphPad Prism software version 4.03 (GraphPad Software, San Diego, Calif.).

### Results

### CD46 and CAR Show Different Expression Patterns on the Surface of Prostate Metastatic Carcinoma Cell Lines

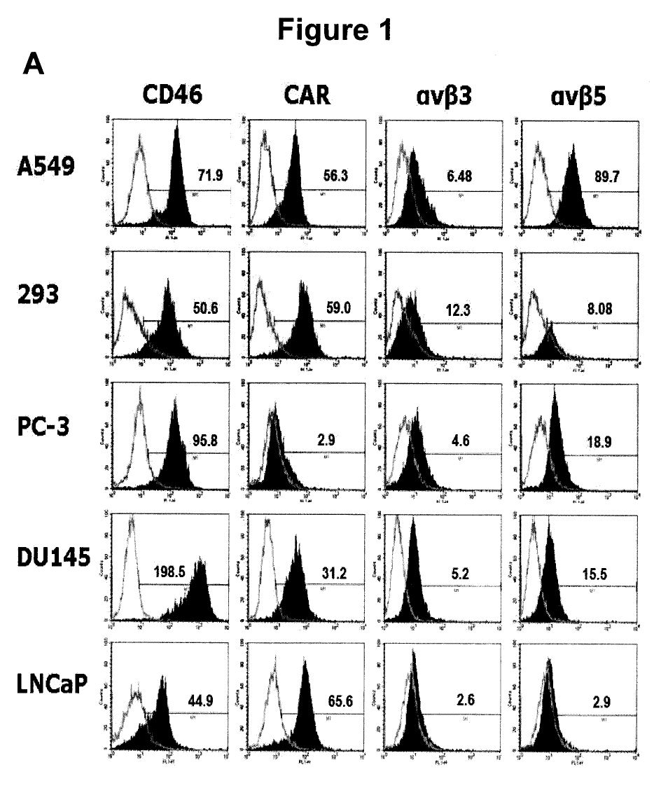

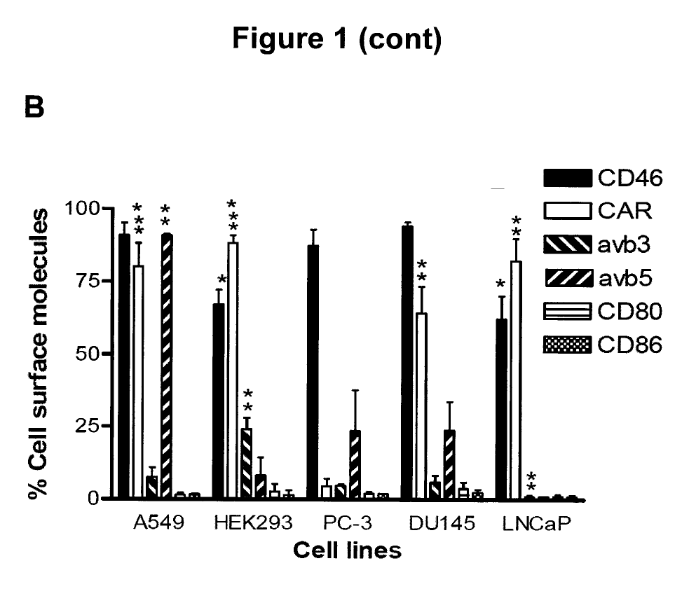

The distribution of cell surface molecules that function as receptors or potential receptors for Ad11 vector in prostate and control cell lines was studied. CAR and CD46 serve as receptors for Ad5 and Ad11, respectively (Tomko et al. 1997. HCAR and MCAR: the human and mouse cellular receptors for subgroup C adenoviruses and group B coxsackieviruses. Proc Natl Acad Sci USA 94(7), 3352-6; Gaggar et al. 2003. CD46 is a cellular receptor for group B adenoviruses. Nat Med 9(11), 1408-12; Segerman et al. 2003. Adenovirus type 11 uses CD46 as a cellular receptor. J Viol 77(17), 9183-91). Cytometric analysis of the immunofluorescence staining of three human prostate cancer cell lines (PC-3, DU 145, and LNCaP) and control cell lines (A549 and HEK-293) showed that the highly tumorigenic cell line PC-3 had a geometric mean value of 95.8 for CD46 expression and only 2.9 for CAR-positive cells. DU 145 manifested the highest amount (198.5) of CD46 expression whereas LNCaP cells had a relatively lower number (44.9) of CD46-positive cells. The geometric mean values for CAR expression in DU 145 and LNCaP cells were 31.2 and 65.6, respectively. A549 and 293 cells derived from lung and kidney had clear expression of CAR (56.3 and 59.0). CD46 showed different expression patterns in these two cell lines: 71.9 for A549 cells and 50.6 for 293 cells. Consequently, DU 145 showed a high degree of expression of CD46 (198.5) as compared with the other cell lines studied, whereas PC-3 cells showed minimal expression of CAR (FIG. 1A).

To verify that interaction of the penton base of RCAd11pGFP vector with cells was also mediated by integrins αᵥβ₃ and αᵥβ₅, the amounts of these molecules on the prostate cancer cell surface were compared with the amounts on A549 and HEK-293 cells. LNCaP cells were cultivated in medium containing R1881 to maintain the androgen dependent property. αᵥβ₃ and αᵥβ₅ integrins were detected at low geometric mean values (2.6 and 2.9, respectively) in LNCaP cells. However, in both androgen-independent PC-3 and DU 145 cell lines, the sum of αᵥβ₃ and αᵥβ₅, integrins was expressed to similar extents: 4.8 and 18.9 for PC-3 cells, and 5.2 and 15.5 for DU 145 cells, respectively (FIG. 1A).

CD80 and CD86 are widely distributed on the surfaces of blood cells and endothelial cells. They are costimulatory molecules involved in T cell activation. It has been reported that all species B adenoviruses use CD80 and CD86 for primary attachment to cells (Short et al. 2006. Members of adenovirus species B utilize CD80 and CD86 as cellular attachment receptors. Virus Res 122(1-2), 144-53). Ad11 of species B:2 has two receptors: CD46 and unknown receptors. Thus, to investigate the possibility and to confirm that either CD80 or CD86 also acts as a receptor for Ad11, these two molecules were included in the receptor screen for Ad11 virus. The expression of CD80 and CD86 was barely detected in all cell lines, with the exception of HEK-293 and DU 145 cells, which exhibited small amounts of CD80 (2.78 and 3.5%) and CD86 (1.62 and 2.5%), respectively. Taken together, these results indicate that CD80 and CD86 might also serve as receptors for Ad11-mediated gene transfer in metastatic prostate cancer cells (FIG. 1B).

The Transduction Efficiency of RCAd11pGFP is Proportional to the Level of Available CD46 Molecules and Integrins

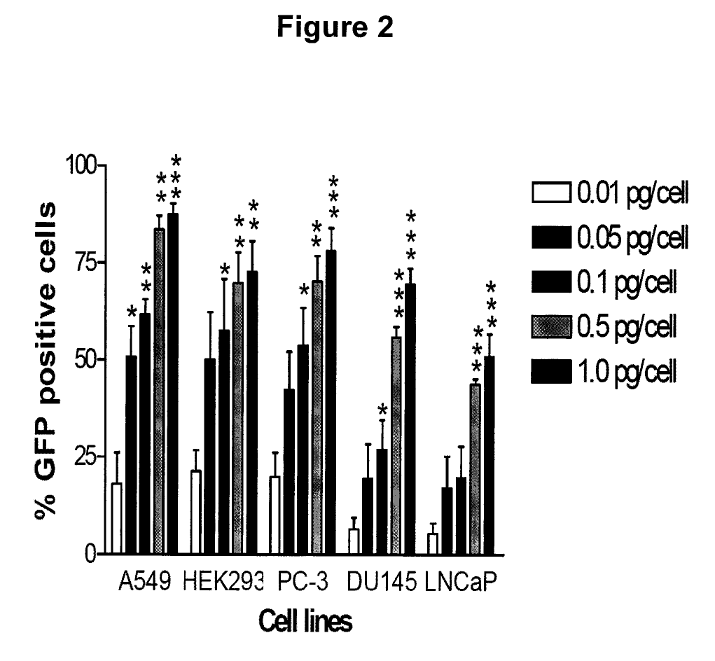

Previous studies have documented the significance of the higher binding activity of Ad11 virus to prostate metastatic cell lines (Zhang et al. 2003. Human adenovirus serotypes 4 and 11 show higher binding affinity and infectivity for endothelial and carcinoma cell lines than serotype 5. J Gen Virol 84(Pt 3), 687-95). To determine whether there was a correlation between viral binding and the number of cell surface molecules that are putative or established primary or secondary receptors, and the extent of correlation with gene transduction efficacy, the three human prostate metastatic cell lines (PC-3, DU 145, and LNCaP cells) and the two control cell lines (A549 and HEK-293) were compared. An RCAd11pGFP vector in which expression of GFP is driven by the cytomegalovirus (CMV) minimal promoter was used. To quantify GFP expression in various prostate carcinoma cells, RCAd11pGFP virus at 0.01, 0.05, 0.1, 0.5, and 1 pg/cell (corresponding to 36, 180, 360, 1800, and 3600 VP/cell or 0.5, 2.5, 5, 25, and 50 TCID₅₀, respectively) was used to infect the various prostate carcinoma cells and flow cytometric assays were used to monitor the levels of expression of GFP. RCAd11pGFP vector was capable of infecting all cell lines tested (FIG. 2), but significantly different levels of GFP expression were observed in cells infected with a low dose of RCAd11pGFP. With RCAd11pGFP at 0.01 and 0.1 pg/cell, less than 5% of the LNCaP cells expressed GFP whereas 10-40% of A549, HEK-293, and PC-3 cells expressed GFP. After infection with the same dose of RCAd11pGFP, DU 145 cells showed higher GFP expression than did LNCaP cells (FIG. 2).

LNCaP cells, which were less efficiently infected with RCAd11pGFP, also expressed CD46, αᵥβ₃, and αᵥβ₅ integrins at lower levels than did PC-3 and DU 145 cells. DU 145 cells were more prone to grow in clusters than the other cells, which might reduce the opportunity of RCAd11pGFP virions to reach all the DU 145 cells.

Transduction is Reduced by Anti-CD46 Antibodies, Ad11prfib, as Well as Antibodies to Ad11prfib, and Integrins

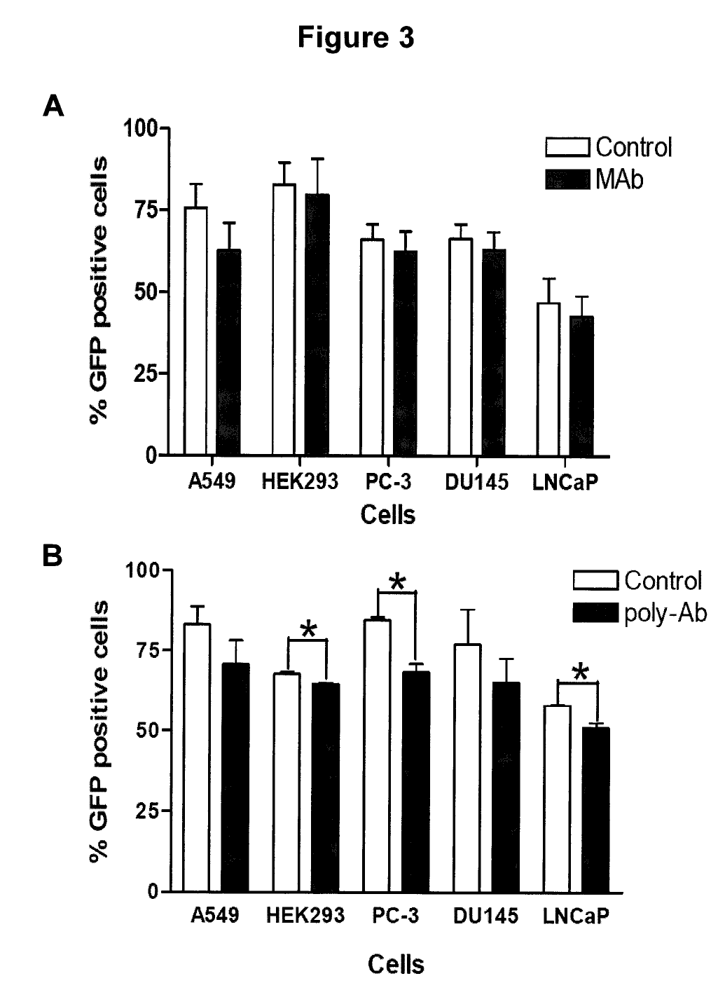

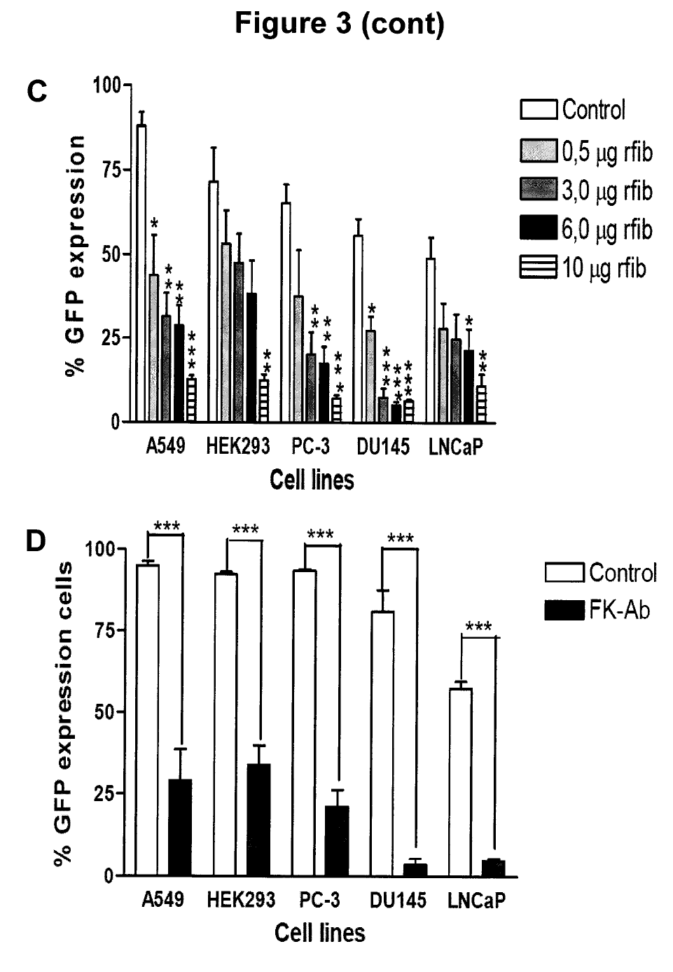

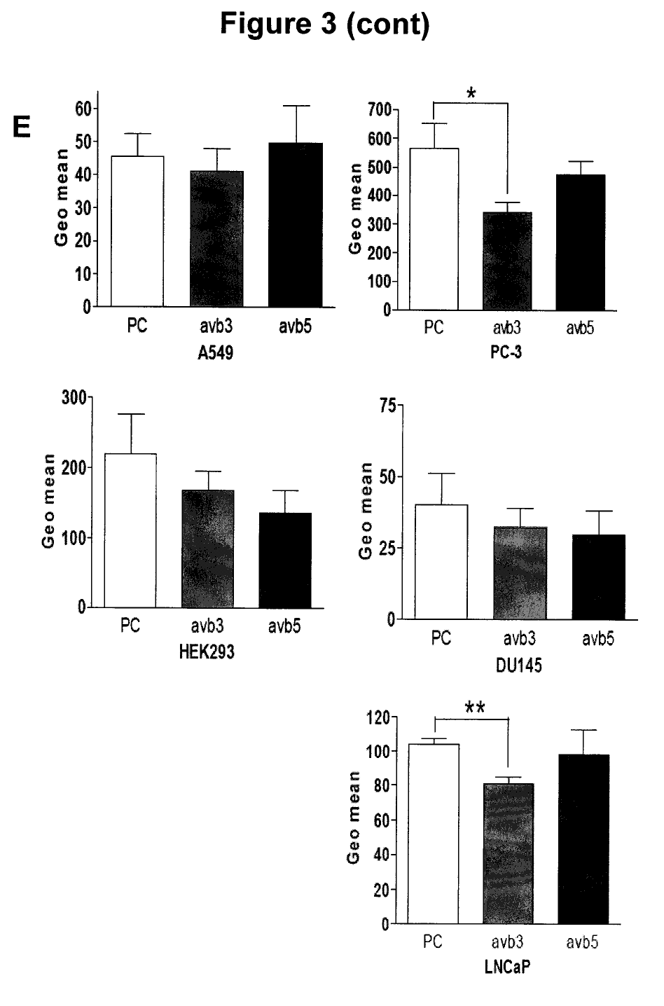

To investigate the importance of CD46 as a primary receptor for RCAd11pGFP vector transduction, blocking experiments with polyclonal and monoclonal anti-CD46 antibodies were performed. The efficiency of infection was assessed by quantification of GFP expression by flow cytometric assay. As shown in FIGS. 3A and 3B, monoclonal anti-CD46 antibody (clone E4.3) was able to inhibit 5% (HEK-293) to 15% of GFP-positive cells, whereas polyclonal anti-CD46 antibody reduced the expression of GFP up to 20% in all cell lines studied. In general, polyclonal anti-CD46 antibody gave stronger inhibition than the monoclonal anti-CD46. GFP expression in PC-3 cells was inhibited approximate 20% when using polyclonal anti-CD46 antibody.

Ad11prfib effectively inhibited transduction mediated by the RCAd11pGFP vector. With Ad11prfib at 0.5 pg/cell, transduction was inhibited 20% in HEK-293 cells and 50% in A549 and DU 145 cells (FIG. 3C). With Ad11prfib at 10 pg/cell, GFP expression could be blocked to 5% in PC-3 and DU 145 cells or 10% in LNCaP, HEK-293, and A549 cells. In addition, the ability to inhibit RCAd11pGFP infection with anti-Ad11prfib antiserum was remarkably efficient in comparison with the results of the CD46 antibody blocking assay (FIG. 3D).

After blocking with anti-integrin antibodies, the extent of GFP expression varied in the various cell lines; however, in general, anti-αᵥβ₃ antibody blocked uptake of virions in all cell lines effectively, whereas anti-αᵥβ₅ antibody inhibited GFP expression in all lines with the exception of A549 cells. This is in line with the high expression of αᵥβ₅ integrin (FIG. 3E). Thus, CD46 may not be the only primary receptor for Ad11 when infecting human prostate cancer cells. Both integrin αᵥβ₃ and integrin αᵥβ₅ affected transduction mediated by the RCAd11pGFP vector.

RCAd11p Replication and Induction of Cytopathic Effect do not Correlate with Cellular p53 Status

Early studies by Hall and colleagues suggested that Ad5 replication required cellular p53 status in the infected cells. This report was not consistent with the later results from Harada and Berk, who claimed that cytopathic effect induced by Ad5 was independent of cellular p53 status. However, there are no reports concerning Ad11 viral replication and cellular p53 level. Therefore, it was investigated whether p53-dependent cell death was needed for induction of RCAd11p cytopathic effect and release of progeny virions from infected cells. Tumor suppressor protein p53 expression in these five cell lines differs greatly: no detectable p53 in PC-3, mutant p53 (p53mut) in DU 145 and LNCaP cells, and wild-type p53 (p53 wt) in A549 and HEK-293 cells have been reported (Graham et al. 1977. Characteristics of a human cell line transformed by DNA from human adenovirus typ^{e} 5. J Gen Virol 36(1), 59-74; Carroll et al. 1993. p53 oncogene mutations in three human prostate cancer cell lines. Prostate 23(2), 123-34; Lehman et al. 1991. p. 53 mutations, ras mutations, and p53-heat shock 70 protein complexes in human lung carcinoma cell lines. Cancer Res 51(15), 4090-6; van Bokhoven et al. 2003. Molecular characterization of human prostate carcinoma cell lines. Prostate 57(3), 205-25).

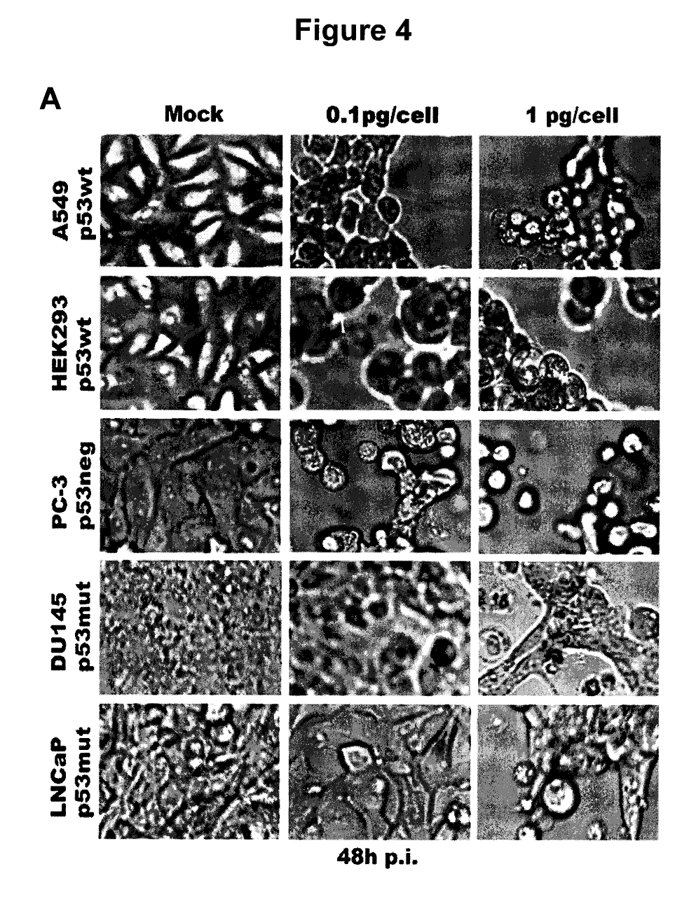

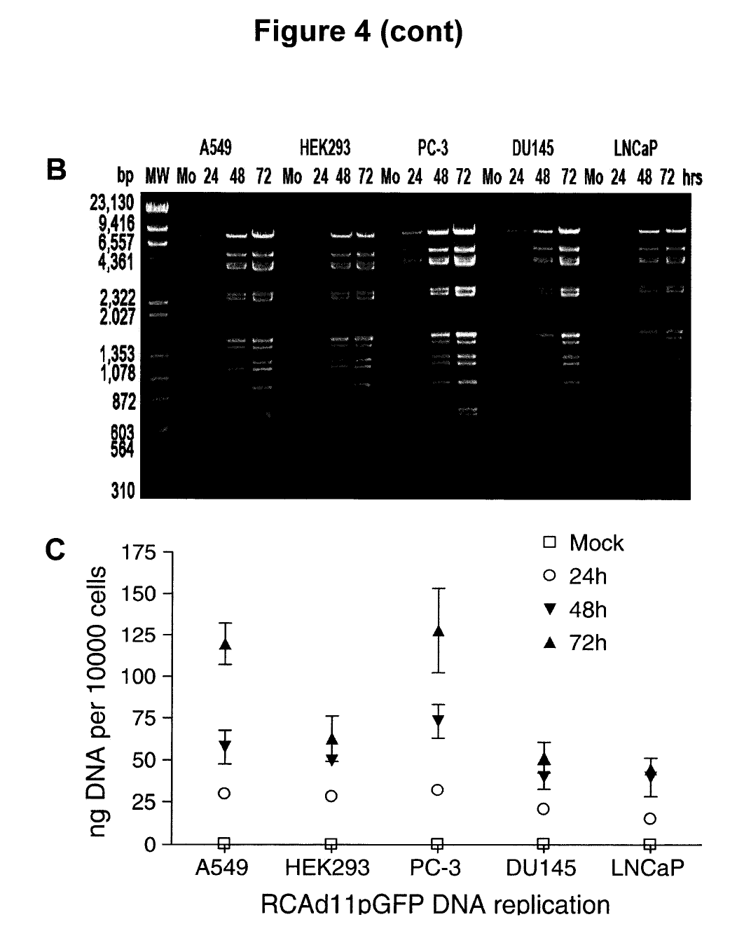

The five cell lines were infected with RCAd11pGFP at 0.1 or 1 pg/cell, corresponding to 5 or 50 TCID50, respectively, and exhibited by phase-contrast microscopy 48 hr postinfection. p53-positive A549 cells and p53-negative PC-3 cells showed obvious cytopathic effect (CPE) by 48 hr at 0.1 pg/cell whereas p53-positive HEK-293 cells and p53-mutant DU 145 and LNCaP cells exhibited CPE with 1 pg/cell by this time (FIG. 4A). Thus, RCAd11pGFP induction of CPE in various cell lines varies widely and dose not correlate with the expression of wild-type p53.

The replication properties of the RCAd11pGFP vector were investigated in prostate cancer cells, and in A549 and HEK-293 cells. The cells were infected with 0.1 pg/cell, and then viral DNA was extracted. Good yields of viral DNA were obtained in PC-3 cells, similar to those in A549 and HEK-293 cells, but the viral DNA was detectable in DU 145 and LNCaP cells at 1 day postinfection. At 2 days postinfection, the level of viral DNA was increased to a high level in PC-3 and DU 145 cells as well as in the control cells (FIGS. 4B and 4C). There was also an increased DNA concentration in LNCaP cells, but the yield was lower than in other cells. The amount of the characteristic restriction fragments revealed that the vector DNA replicated in PC-3 cells as efficiently as in A549 cells during the observation period. Late expression of viral proteins in the prostate cancer cell lines was also compared and it was found that the yield of hexons correlated with the cytotoxicity results: hexon protein was highly expressed in PC-3 cells but was just detectable in LNCaP cells. An intermediate amount of hexons was observed in DU 145 cells (Table 1). Therefore, DNA replication, protein expression, and oncolytic effects of RCAd11pGFP were not dependent on the p53 level of the target cells but, rather, on the intrinsic properties of targeting tumor cells.

TABLE 1
Transduction and cytolytic effect mediated by RCAd11pGFP are independent of p53 level in targeting cell lines.

| | | | | % GFP | DNA | CPE with | Viral | Cytotoxic assay | | |
|---|---|---|---|---|---|---|---|---|---|---|
| | p53 gene | % CD46 | % CAR | expression at | replication | 0.1 pg/cell | protein | at day 6 p.i. | One step replication | |
| Cell type | status^{a} | expression | expression | 1 pg/cell, 48 h p.i. | (ng/10⁴cells)^{b} | at 48 h p.i. | level^{c} | pg/cell | Day 1 | Day2 |
| A549 | p53wt | 90.1 | 80.3 | 85 | 113.5 | ++++ | ++++ | 0.0001 | 10⁶ | 10⁸ |
| HEK293 | p53wt | 67.1 | 88.3 | 70 | 60 | ++ | +++ | 0.001 | 5 × 10⁵ | 5 × 10⁷ |
| PC-3 | p53neg | 87.4 | 4.7 | 80 | 110 | ++++ | ++++ | 0.0001 | 10⁶ | 10⁸ |
| DU145 | p53mut | 94.1 | 64.3 | 67 | 50 | ++ | +++ | 0.01 | 5 × 10⁵ | 5 × 10⁷ |
| LNCaP | p53mut | 72.4 | 82.7 | 50 | 48 | ++ | ++ | 0.01 | 10⁴ | 10⁶ |

Abbreviations: CAR, coxsackievirus-adenovirus receptor; CPE, cytopathic effect; GFP, green fluorescent protein; p53mut, mutant p53; p53neg, negative for p53; p53wt, wild-type p53; p.i., postinfection.
^{a}p53 gene status was determined from the literature: Carroll et al., 1993; Hall et al., 1998; Lehman et al., 1991; van Bokhoven et al., 2003.
^{b}Qualitative assessment of viral DNA isolated from RCAd11pGFP vector at 72 hr postinfection.
^{c}Determined by Western blot analysis of viral protein expression levels

RCAd11pGFP Vector can Spread Efficiently from Cell to Cell in p53⁻ Pc-3 and P53⁺ A549 Cells

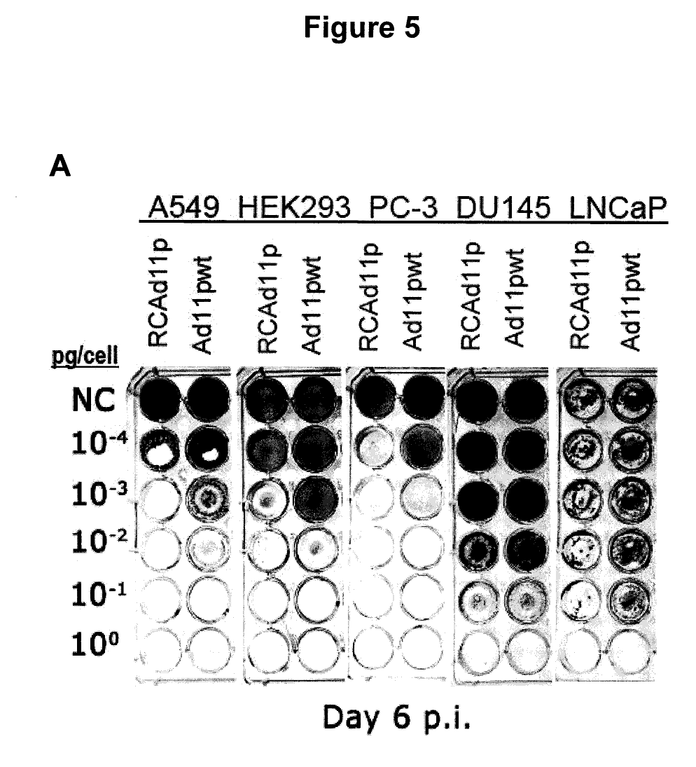

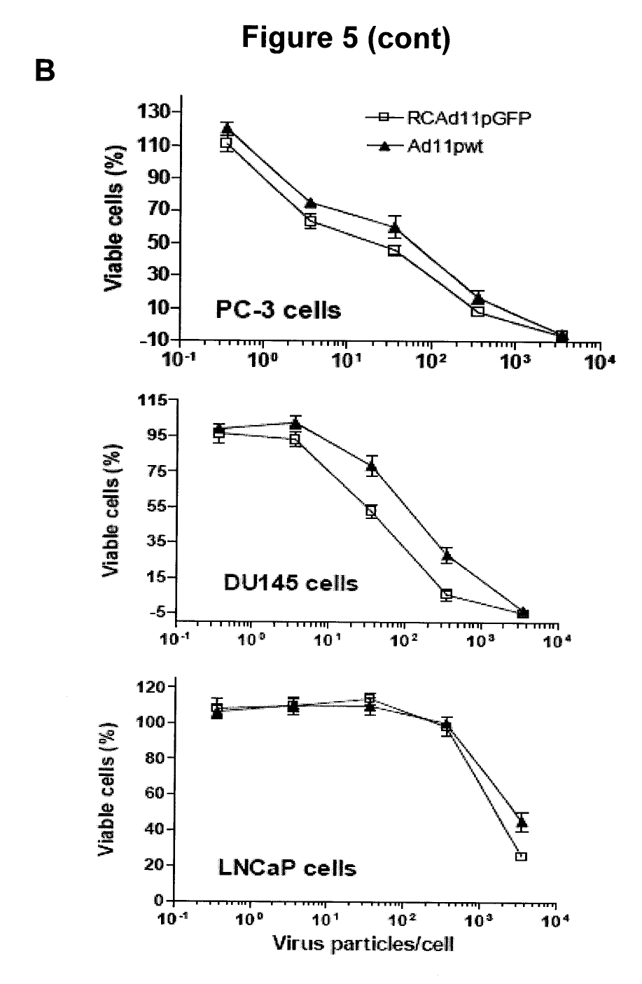

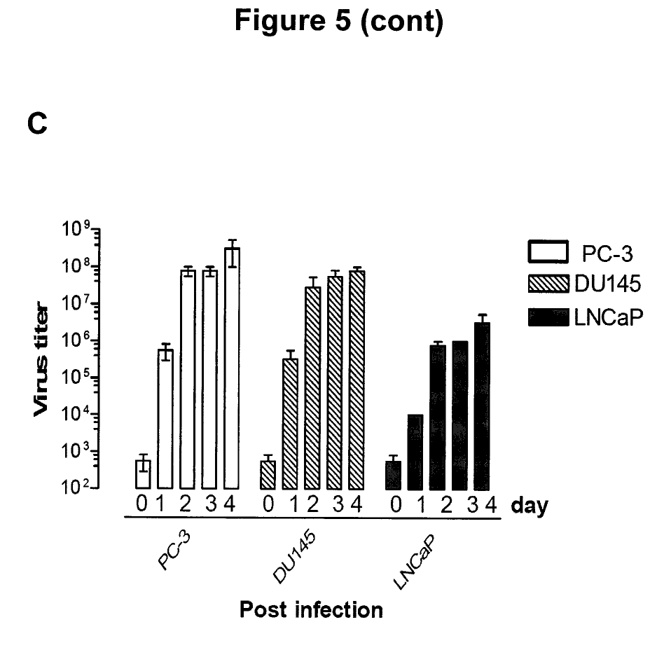

To achieve oncolytic efficacy, a replication-competent tumor therapy vector should be able to spread rapidly from tumor cell to tumor cell. The high expression of GFP mediated by the RCAd11pGFP vector should lead to rapid cell lysis and vector release, resulting in an increasing infection-release-reinfection cycle. To evaluate the likelihood of this reasoning, a toxicity assay on prostate cancer cells was performed, A549 cells, and HEK-293 cells infected with RCAd11pGFP and wild-type Ad11p. As shown in FIG. 5A and Table 1, cells in 24-well plates were infected with four 10-fold dilutions of the two viruses, starting at 1 pg/cell. The negative control had no virus added. At 6 days postinfection, the monolayers were stained with crystal violet as described in Materials and Methods. Cells infected at the highest multiplicity of infection (1 pg/cell) were rapidly destroyed. However, in cells infected with 0.0001 pg/cell, the virus must initiate one or more cycles of infection to cause detectable cytopathic effect. RCAd11pGFP replicated 2 logs more efficiently in PC-3 cells than in LNCaP cells. These data indicate that of the five cell lines tested, the most efficient growth of RCAd11pGFP was in PC-3 and A549 cells (Table 1). Furthermore, no great difference in the growth patterns of RCAd11pGFP and wild-type Ad11p was observed (FIG. 5A).

RCAd11pGFP Significantly Reduces Proliferation of PC-3 and DU 145 Cells, but not that of LNCaP Cells

To study the effect of RCAd11p vector and Ad11pwt on cell viability and proliferation, the three prostate metastatic cell lines were infected with the viruses. As shown in FIG. 5B, viable cells and proliferation of PC-3, DU 145, LNCaP cells were obviously reduced at 3.6, 36, and 360 VP/cell, respectively. No distinct difference between RCAd11pGFP and Ad11pwt was observed in the cell viability assay. PC-3 and DU 145 cells were more sensitive to RCAd11pGFP infection than were LNCaP cells. The strongest effect was observed at 1 pg/cell (3600 VP/cell), which caused the death of more than 90% of PC-3 and DU 145 cells and 80% of LNCaP cells at 6 days postinfection.

RCAd11pGFP Vector Multiplies 100 Times more Efficiently in PC-3 Cells than in LNCaP Cells

The replicative capacity of RCAd11pGFP was determined by a one-step growth assay. Cells were infected with 0.2 pg of virus per cell, and the total amount of vector was harvested 24, 48, 72, and 96 hr postinfection and measured by titration in HEK-293 cells. The viral titer was higher in PC-3 cells than in the other two cell lines; it was 5 logs higher after 96 hr of incubation. In DU 145 cells it was slightly less over the same time period, reaching 4.5 logs. In contrast, the viral titer of vector in LNCaP cells was 3 logs higher than in the original inoculum. Thus, RCAd11p vector did grow 100-fold better in PC-3 cells than in LNCaP cells. With the same infectious dose and the same incubation period, the different capacity of RCAd11pGFP to grow in prostate metastatic cells suggested that the natural property of the vector and the tumorigenicity of targeting cells are decisively important for viral replication (FIG. 5C).

### Antitumor Effect of RCAd11p Vector in PC-3 Subcutaneous Tumor

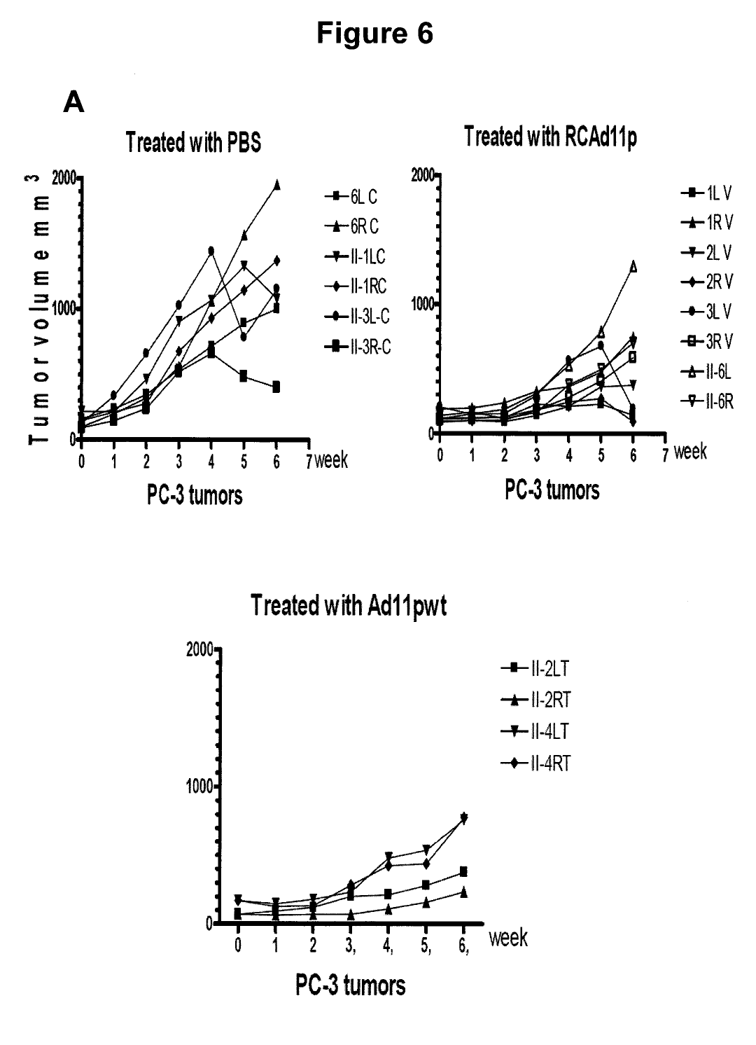

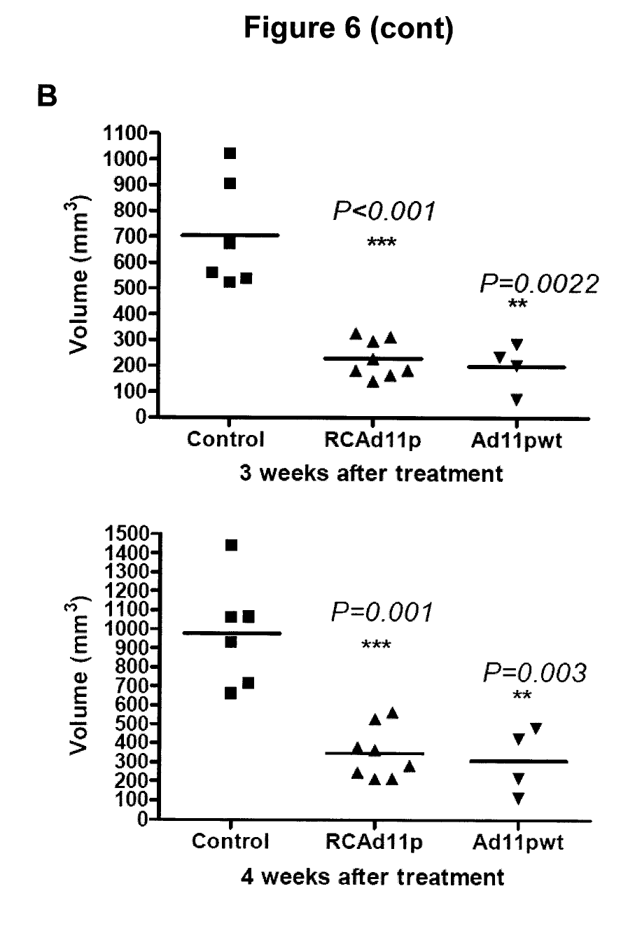

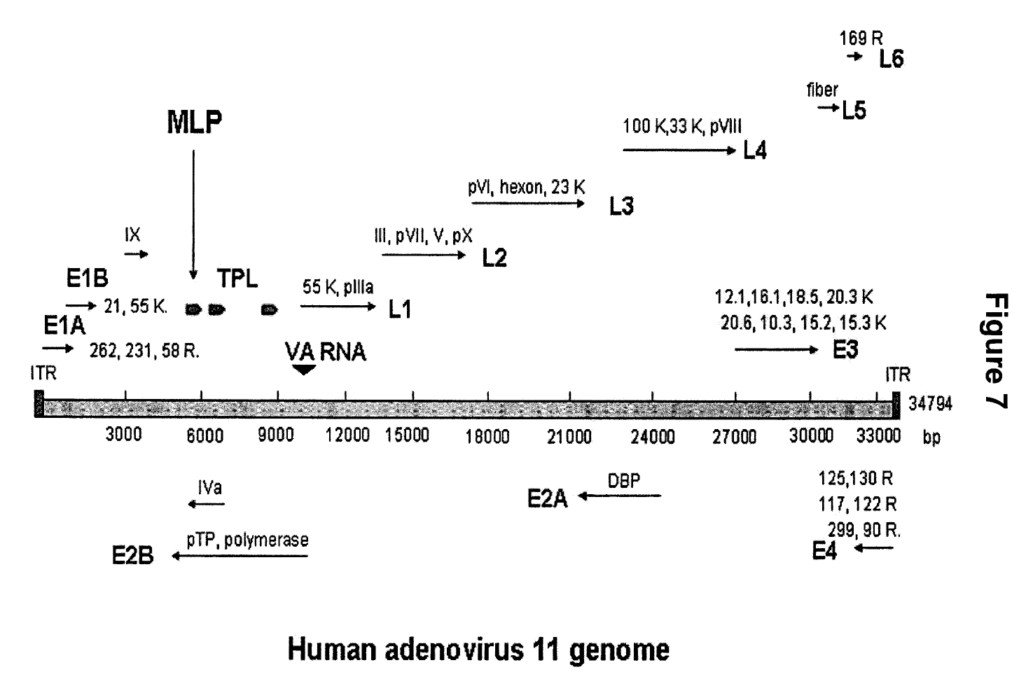

The weight of each animal and the size and shape of tumors were measured and photographed before injection with virus and before euthanasia. Athymic BALB/c mice 4-5 weeks of age with PC-3 subcutaneous tumor were treated with either RCAd11p vector or Ad11pwt virus once via intratumoral injection. Control group tumors were treated with PBS. All inoculated or control mice survived the 6-week experimental period. After treatment, the group treated with RCAd11p vector or Ad11pwt virus demonstrated significant growth inhibition of the PC-3 tumors for up to 42 days as compared with mock-treated PC-3 tumors (FIGS. 6A and 6B); which were 2.5- to 3.5-fold greater in size, respectively, than the tumors treated with RCAd11p or Ad11pwt.

### Discussion

The lethal phenotypes of human prostate cancer are characterized by progression from androgen dependence to androgen independence, with a propensity to form bone metastases. In almost 80% of prostate cancer cases, the bone is colonized and a characteristic osteoblast reaction is elicited. Despite therapeutic advances involving combinations of chemotherapy, radiation treatment, and immunotherapy, most patients with metastatic prostate cancer still die within a few years (Hickey et al. 1988. Failure of chemotherapy to prolong life in patients with metastatic prostate cancer who have failed androgen deprivation. Urology 31(1), 38-40; Coleman, 2006. Clinical features of metastatic bone disease and risk of skeletal morbidity. Clin Cancer Res 12(20 Pt 2), 6243s-6249s; Mike et al. 2006. Chemotherapy for hormone-refractory prostate cancer. Cochrane Database Syst Rev (4), CD005247). Gene therapy mediated by Ad5 has shown less efficient transduction of metastatic prostate carcinoma and clinical bladder cancer because these cells lack the Ad5 primary receptor (CAR) (Li et al. 1999. Loss of adenoviral receptor expression in human bladder cancer cells: a potential impact on the efficacy of gene therapy. Cancer Res 59(2), 325-30; Rauen et al. 2002. Expression of the coxsackie adenovirus receptor in normal prostate and in primary and metastatic prostate carcinoma: potential relevance to gene therapy. Cancer Res 62(13), 3812-8; Sachs et al. 2002. Integrin alpha(v) and coxsackie adenovirus receptor expression in clinical bladder cancer. Urology 60(3), 531-6). There is increasing awareness that the development of novel alternative adenoviral vectors that target non-CAR receptors with low seroprevalence may improve survival rates among patients with this form of cancer. To address this end, a genetically engineered RCAd11p vector was assessed and it was explored whether this virus could be efficiently expressed in cell lines derived from metastatic prostate cancer.

The present inventors have described in vitro and in vivo characterization of a novel replication-competent Ad11 vector that expresses the entire viral genome and, in addition, a GFP expression cassette. This vector contains both the E1A and E1B 55k open reading frames (ORFs). Ad5 vector with the E1B55k ORF deleted has been found to show tumor-specific replication (Bischoff et al. 1996. An adenovirus mutant that replicates selectively in p53-deficient human tumor cells. Science 274(5286), 373-6). However, in the latter report there is no information given about the efficiency of viral replication in the presence of the E1B55k ORF. The role of the E1A and E1B ORFs were taken into account and these ORFs were retained in the RCAd11p vector, with the purpose of investigating the natural properties of the vector, such as transduction and oncolysis.

Integrins αᵥβ₃ and αᵥβ₅ are secondary receptors for Ad2 and Ad5 (Wickham et al. 1993. Integrins alpha v beta 3 and alpha v beta 5 promote adenovirus internalization but not virus attachment. Cell 73(2), 309-19). αᵥβ₃ and αᵥβ₅ integrins apparently showed higher expression in PC-3 and DU 145 cells than in LNCaP cells. With monoclonal antibody to these integrins (MAB1976) recognizing the vitronectin receptor in αᵥβ₃complex, an RGD-directed adhesion receptor, transduction mediated by the Ad11 vector was reduced by 10 and 40% for A549 and PC-3 cells, respectively. The presently results suggest that these integrins play an important role in infection of metastatic prostate cancer cells by Ad11.

Thus, transduction by RCAd11pGFP relies on both CD46 and integrins on prostate cancer cells. With high amounts of CD46 and integrins on PC-3 and DU 145 cells (Table 1), GFP expression was higher in these cells than in LNCaP cells, which appear to express relatively low numbers of such receptor molecules. The transduction efficiency was also dose dependent, as with other viral vectors (Kustikova et al. 2003. Dose finding with retroviral vectors: correlation of retroviral vector copy numbers in single cells with gene transfer efficiency in a cell population. Blood 102(12), 3934-7).

The CD46 molecule consists of four domains: CCP1, CCP2; CCP3, and CCP4 (also designated SCR1, SCR2, SCR3, and SCR4). It was previously demonstrated that Ad5/Ad35 chimeric virus requires CCP2 (SCR2) for infection (Gaggar et al. 2005. Localization of regions in CD46 that interact with adenovirus. J Virol 79(12), 7503-13; Stone et al. 2005. Development and assessment of human adenovirus type 11 as a gene transfer vector. J Virol 79(8), 5090-104). In this study, the CD46-specific mAb (clone E4.3), which recognizes the SCR1 domain, only partially blocked the attachment of Ad11 virions to prostate cancer cells. Some epitopes recognized by other anti-CD46 mAbs (MEM258 and M177) can more efficiently block Ad35 binding to the cell surface, as reported by Fleischli et al. (2005. The distal short consensus repeats 1 and 2 of the membrane cofactor protein CD46 and their distance from the cell membrane determine productive entry of species B adenovirus serotype 35. J Virol 79(15), 10013-22). Furthermore, the crystal structure of the complex between Ad11prfib and CD46 has been elucidated, and this indicated that Ad11prfib interacts with both the SCR1 and SCR2 domains of the CD46 molecule (Persson et al. 2007. Adenovirus type 11 binding alters the conformation of its receptor CD46. Nat Struct Mol Biol 14(2), 164-6). Ad11 binding reshapes the conformation of its receptor so that the bent surface structure straightens into an elongated rod. All these results might explain why neither anti-CD46 mAb nor polyclonal antibodies can efficiently block viral infection. The fiber is the main viral ligand for attachment to prostate cancer cell lines, because viral infectivity was almost totally inhibited by Ad11prfib or anti-rfib antibody.

The three classic prostate cancer cell lines express different amounts of tumor suppressor protein (p53). PC-3 cells do not express any detectable p53, whereas LNCaP cells and DU 145 cells produce mutant p53 (Carroll et al. 1993. p53 oncogene mutations in three human prostate cancer cell lines. Prostate 23(2), 123-34; van Bokhoven et al. 2003. Molecular characterization of human prostate carcinoma cell lines. Prostate 57(3), 205-25). PC-3 cells are derived from a highly tumorigenic metastatic prostate carcinoma.

Both the PC-3 and the DU 145 cell lines are androgen independent whereas LNCaP cells are androgen-dependent metastatic cells. Regarding clinical treatment, androgendependent prostate cancer is sensitive to chemotherapy. When prostate cancer cells develop androgen independence, all kinds of treatment become ineffective. Interestingly, the replicative capacity of Ad11 in PC-3 and DU145 cells was 100-fold more efficient than in LNCaP cells (Table 1).

The results present here demonstrate that the RCAd11 vector may be a promising tool for the treatment of metastatic prostate cancer. Early reports suggested that ONYX-015 (also called d11520), based on the Ad5 vector with deletion of the E1B 55k ORF, did selectively kill tumor cells harboring a defective p53 gene (Bischoff et al. 1996. An adenovirus mutant that replicates selectively in p53-deficient human tumor cells. Science 274(5286), 373-6; Heise et al. 1997. ONYX-015, an E1B gene-attenuated adenovirus, causes tumor-specific cytolysis and antitumoral efficacy that can be augmented by standard chemotherapeutic agents. Nat Med 3(6), 639-45); however, subsequent reports have shown that ONYX-015 replicates and kills tumor cells with wild-type p53 at least as efficiently as it kills cells lacking p53 (Hall et al. 1998. p53-dependent cell death/apoptosis is required for a productive adenovirus infection. Nat Med 4(9), 1068-7; Rothmann et al. 1998. Replication of ONYX-015, a potential anticancer adenovirus, is independent of p53 status in tumor cells. J Virol 72(12), 9470-8; Dix et al. 2000. Efficient induction of cell death by adenoviruses requires binding of EJB55k and p53. Cancer Res 60(10), 2666-72).

The present results based on an RCAd11p vector, demonstrate that Ad11 vector carrying the E1B 55k ORF replicates efficiently in tumor cells expressing wild-type p53 and expressing mutant p53 as well as lacking p53 expression.

Previously it was reported that replication of the Ad5 wildtype or d11520 strain was 4- to 24-fold lower in A549 than in PC-3 cells (Harada and Berk, 1999. p53-Independent and-dependent requirements for E1B-55K in adenovirus type S replication. J Virol 73(7), 5333-44). Low expression of CAR in the highly tumorigenic PC-3 cell line was observed in this study, suggesting that the CAR-targeting vector, Ad5, might not be suitable for gene therapy in such tumor types. CD46 was highly expressed on the surface of PC-3 cells and RCAd11pGFP infected PC-3 cells easily and caused high expression of the GFP gene. It has previously been reported that wildtype Ad11p shows higher binding affinity for two prostate cell lines than does Ad5 (Zhang et al. 2003. Human adenovirus serotypes 4 and 11 show higher binding affinity and infectivity for endothelial and carcinoma cell lines than serotype 5. J Gen Virol 84(Pt 3), 687-95). In the present study, RCAd11p clearly showed higher infectivity in prostate cancer cells, all of which express CD46 at high levels on their surface. The CD46 molecule is widely distributed on the cell surface of nucleated human cells (Liszewski et al. 2005. Emerging roles and new functions of CD46. Springer Semin Immunopathol 27(3), 345-58). The transduction assay mediated by interaction between RCAd11p vector and CD46 showed that this vector efficiently binds to cells, is taken up by them, and enters the nucleus.

Strains of the Edmonston lineage of measles virus also use CD46 as primary cellular receptor. The killing of tumor cells by measles virus has been found to be dependent on the density of CD46 on the cell surface (Anderson et al. 2004. High CD46 receptor density determines preferential killing of tumor cells by oncolytic measles virus. Cancer Res 64(14), 4919-26). The novel Ad11 vector showed a different way of killing target cells: first, viral transduction efficiency relied on the density of cell surface receptors, where the amount of GFP expression was correlated with the number of CD46 molecules on the cell surface; second, the oncolytic function of the vector was dependent on tumor cells and the vector construct. Not one of the two factors is dispensed to complete the oncolytic infection cycle. In the highly tumorigenic PC-3 cells, RCAd11p vector showed a high degree of replication, whereas in DU 145 cells the replicative efficacy of RCAd11p vector was better than in androgen-dependent LNCaP cells. Gene transfer to LNCaP cells mediated by the Ad11p vector was as efficient as for other cells, but the production of oncolytic vector particles is limited in LNCaP cells. Therefore, an Ad11 vector that carries a virus-specific promoter and a GFP expression cassette was generated. With this construct, it was possible to evaluate the transduction and replicative capacity of this vector. The benefit of using RCAd11p vector is that this virus vector allows efficient transduction and has a high replicative capacity in most tumorigenic cells.

Here it is demonstrated those metastatic prostate carcinoma cell lines support RCAd11pGFP replication and the production of infectious progeny, with yields from 6 to 8 logs. It is also demonstrated that RCAd11pGFP replicates in PC-3 cells most efficiently. The PC-3 cell line was highly tumorigenic, androgen independent, and tolerant of chemotherapy. It is further demonstrated that the oncolytic RCAd11p vector exhibits considerable antitumor efficacy in xenografted nude mice. The growth of PC-3 tumors was limited by injection of either RCAd11p or Ad11pwt, whereas control tumors without virotherapy grew rapidly. In a comparison of treated and untreated tumors, a significant difference (p<0.001) was observed at 3 or 4 weeks postinfection. Moreover, it is demonstrated that the mice subjected to virotherapy were healthy whereas the untreated mice clearly suffered from the effect of tumors. The results demonstrate that the RCAd11p vector enhanced in vivo production of virus in initially infected tumor cells with subsequent dissemination to neighboring tumor cells. Therefore, RCAd11p efficiently exerts an oncolytic effect in xenografted nude mice.

In summary, the RCAd11p vector provides advantages for gene therapy of both androgen-independent and androgen-dependent late-stage metastatic prostate cancer, because this vector transfers genes efficiently to several human cell lines but shows high-level replication in metastatic prostate cells in bone. The oncolytic capacity of the RCAd11p vector relies on the type of tumor rather than on the presence or absence of the E1B55k ORF in the vector. These findings indicate that the RCAd11 vector—particularly under the control of the viral early region 1 (E1A) promoter—has potential for the treatment of highly tumorigenic cells. Consequently, gene therapy based on RCAd11p may be useful in the future treatment of metastatic prostate carcinomas.

### Example 2

### Effect of Replication-Competent Ad11p Vector on the Growth of Human Colon T84 Tumours in Nude Mice

In Vivo Oncolytic Model with RCAd11p Vector.

T84 cells derived from a lung metastasis of a colorectal adenocarcinoma and HT 29 derived from colorectal adenocarcinoma were used in the experiment. 10⁷ T84 cells or H-29 cells in 0.2 ml were subcutaneously transplanted into left and right flank region of Balb/c nude mice, the control group mice were injected with only PBS. As colon tumor grew up to at least 75 mm³ 3 weeks after injection, 50 μg of RCAd11pGFP/tumor was injected into mice with intratumoral administration. Tumor volume was recorded weekly. The mice were sacrificed approximately 6 weeks after viral injection.

As can be seen from the results presented in FIG. 10, treatment with the adenovirus vector RCAd11pGFP reduced the growth of the transplanted colon tumours to a similar extent as treatment with wild-type Ad11p, demonstrating that the recombinant adenovirus vector carrying an insertion of a heterologous nucleic acid fragment encoding GFP maintained replication competence, infectivity and oncolytic activity.

1. A recombinant replication-competent Ad11p adenovirus vector, comprising an isolated nucleic acid comprising a sequence having at least 90%, preferably at least 95%, more preferably at least 98%, and most preferably at least 99% sequence identity to the sequence set forth in SEQ ID NO:1, further comprising one or more inserts of a heterologous nucleic acid fragment, wherein said heterologous nucleic acid fragment is cloned into a position corresponding to between nucleotide 247 and nucleotide 568, or into a position corresponding to between nucleotide 27836 and nucleotide 29526, of SEQ ID NO: 1.

2. A recombinant replication-competent Ad11p adenovirus vector according to claim 1, wherein said heterologous nucleic acid fragment is cloned into a position corresponding to between nucleotide 247 and nucleotide 568, preferably between nucleotide 382 and nucleotide 568, more preferably between nucleotide 382 and nucleotide 479, most preferably between nucleotide 436 and nucleotide 479 of SEQ ID NO:1.

3. A recombinant replication-competent Ad11p adenovirus vector according to claim 1, wherein said heterologous nucleic acid fragment is cloned into a position corresponding to between nucleotide 27836 and nucleotide 29526, preferably between nucleotide 27836 and nucleotide 28356, between nucleotide 28356 and nucleotide 29481, between nucleotides 28901 and nucleotide 28919, or between nucleotide 28920 and nucleotide 29526, of SEQ ID NO:1.

4. A recombinant replication-competent Ad11p adenovirus vector according to claim 3, wherein said heterologous nucleic acid fragment is cloned into a position corresponding to between nucleotide 27836 and nucleotide 28356.

5. A recombinant replication-competent Ad11p adenovirus vector according to claim 1, wherein said insert has a size of between 100 and 5,000 base pairs, such as a between 500 to 2,000 base pairs.

6. A recombinant replication-competent Ad11p adenovirus vector according to claim 1, wherein said insert encodes a siRNA, a shRNA, or a micro RNA.

7. A recombinant replication-competent Ad11p adenovirus vector according to claim 1, wherein the expression of said heterologous nucleic acid fragment is under the control of a heterologous enhancer and/or promoter.

8. A recombinant replication-competent Ad11p adenovirus vector according to claim 7, wherein said heterologous enhancer and/or promoter is selected from heterologous viral promoters, and mammalian tissue specific promoters.

9. A recombinant replication-competent Ad11p adenovirus vector according to claim 1, wherein the expression of said heterologous nucleic acid fragment is under the control of adenoviral endogenous gene expression machinery or an adenoviral endogenous enhancer and/or promoter.

10. A recombinant replication-competent Ad11p adenovirus vector according to claim 1, wherein said heterologous nucleic acid fragment encodes a protein selected from the group consisting of: a viral protein, an antigenic determinant of a pathogenic organism, a tumor-specific antigen, a human protein, a cytokine.

11. A recombinant replication-competent Ad11p adenovirus vector according to claim 1, wherein said heterologous nucleic acid fragment encodes a viral protein selected from the group consisting of ADP, E1A, and p300.

12. A recombinant replication-competent Ad11p adenovirus vector according to claim 1, wherein said heterologous nucleic acid fragment encodes a human protein selected from the group consisting of: Rb, CFTR, p16, p21, p27, p57, p73, C-CAM, APC, CTS-1, zacl, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, fus, interferon α, interferon β, interferon γ, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NTS, ApoAI, ApoAIV, ApoE, RaplA, cytosine deaminase, Fab, ScFv, BRCA2, zacl, ATM, HIC-1, DPC-4, FHIT, PTEN, ING1, NOEY1, NOEY2, OVCA1, MADR2, 53BP2, IRF-1, Rb, zacl, DBCCR-1, rks-3, COX-1, TFPI, PGS, Dp, E2F, ras, nzyc, neu, raf, erb, fins, trk, ret, gsp, hst, abl, VEGF, FGF, PEDF, thrombospondin, BAI-1, GDAIF, and MCC.

13. A recombinant replication-competent Ad11p adenovirus vector according to claim 1, wherein said heterologous nucleic acid fragment encodes a reporter protein selected from the group consisting of: a fluorescent protein, a luminescent protein, and an enzyme.

14. A recombinant replication-competent Ad11p adenovirus vector according to claim 13, wherein said reporter protein is selected from the group consisting of green fluorescent protein, enhanced green fluorescent protein, yellow fluorescent protein, blue fluorescent protein, cyan fluorescent protein, red fluorescent protein, luciferase, β-galactosidase, and chloramphenicol acetyltransferase.

15. A pharmaceutical composition comprising a recombinant replication-competent adenovirus vector according to claim 1, and a suitable excipient.

16-21. (canceled)

22. A method of treating a mammalian patient having a cancer disease, said method comprising administering to the patient a pharmaceutical comprising an isolated nucleic acid comprising a sequence having at least 90%, preferably at least 95%, more preferably at least 98%, and most preferably at least 99% sequence identity to the sequence set forth in SEQ ID NO:1, further comprising one or more inserts of a heterologous nucleic acid fragment, wherein said heterologous nucleic acid fragment is cloned into a position corresponding to between nucleotide 247 and nucleotide 568, or into a position corresponding to between nucleotide 27836 and nucleotide 29526, of SEQ ID NO: 1.

23. The method according to claim 22, wherein the pharmaceutical composition is administered locally to a tumour site.

24. The method according to claim 22, wherein the pharmaceutical composition is administered intravesically or intraductally.

25. The method according to claim 22, wherein the pharmaceutical composition is administered by intravenous injection.

26. The method according to claim 22, further comprising administering a chemotherapeutic agent to the patient.

27. The method according to claim 22, further comprising treating the patient with radiation therapy.

28. (canceled)

29. A mammalian cell comprising a recombinant replication competent adenovirus vector according to claim 1.

30. A method of expressing a protein or gene product or an expression product which comprises infecting or transfecting a cell in vitro with a recombinant replication competent adenovirus vector according to claim 1 and optionally extracting, purifying or isolating the protein, gene product or expression product from the cell.

31. (canceled)

32. The method according to claim 22, wherein said disease is selected from the group consisting of: a viral infectious disease, a bacterial infectious disease, a cancer disease, vascular disease, a cardiovascular disease, an immunological disease, a liver disease, and a parasitic disease.

33. The method according to claim 22, wherein said viral infectious disease is selected from the group consisting of viral diseases caused by HCV, HBV, HIV, and influenza A virus.

34. The method according to claim 22, wherein said bacterial infectious disease is selected from the group consisting of tuberculosis, and septic chock.

35. The method according to claim 22, wherein said cancer disease is selected from the group consisting of: lung cancer, breast cancer, ovarian cancer, prostate cancer, colorectal cancer, bladder cancer, kidney cancer, haematological malignancies, liver cancer, pancreatic cancer, tumours of neural origin, head and neck cancer, and osteosarcoma.


SEQUENCE LISTING (1 sequence)

SEQ ID NO: 1 - DNA, 34794 bp, Adenoviridae
  misc_feature (1)...(34794): Human adenovirus 11
catcatcaataatataccttatagatggaatggtgccaatatgtaaatgaggtgattttaaaaagtgtggatcgtgtggtgattggctgtggggttaacggctaaaaggggcggtgcgaccgtgggaaaatgacgttttgtgggggtggagtttttttgcaagttgtcgcgggaaatgtgacgcataaaaaggcttttttctcacggaactacttagttttcccacggtatttaacaggaaatgaggtagttttgaccggatgcaagtgaaaattgttgattttcgcgcgaaaactgaatgaggaagtgtttttctgaataatgtggtatttatggcagggtggagtatttgttcagggccaggtagactttgacccattacgtggaggtttcgattaccgtgttttttacctgaatttccgcgtaccgtgtcaaagtcttctgtttttacgtaggtgtcagctgatcgctagggtatttatacctcagggtttgtgtcaagaggccactcttgagtgccagcgagaagagttttctcctctgcgccggcagtttaataataaaaaaatgagagatttgcgatttctgcctcaggaaataatctctgctgagactggaaatgaaatattggagcttgtggtgcacgccctgatgggagacgatccggagccacctgtgcagctttttgagcctcctacgcttcaggaactgtatgatttagaggtagagggatcggaggattctaatgaggaagctgtaaatggcttttttaccgattctatgcttttagctgctaatgaagggttagaattagatccgcctttggacacttttgatactccaggggtaattgtggaaagcggtacaggtgtaagaaaattacctgatttgagttccgtggactgtgatttgcactgctatgaagacgggtttcctccgagtgatgaggaggaccatgaaaaggagcagtccatgcagactgcagcgggtgagggagtgaaggctgccaatgttggttttcagttggattgcccggagcttcctggacatggctgtaagtcttgtgaatttcacaggaaaaatactggagtaaaggaactgttatgttcgctttgttatatgagaacgcactgccactttatttacagtaagtgtgtttaagttaaaatttaaaggaatatgctgtttttcacatgtatattgagtgtgagttttgtgcttcttattataggtcctgtgtctgatgctgatgaatcaccatctcctgattctactacctcacctcctgagattcaagcacctgttcctgtggacgtgcgcaagcccattcctgtgaagcttaagcctgggaaacgtccagcagtggaaaaacttgaggacttgttacagggtggggacggacctttggacttgagtacacggaaacgtccaagacaataagtgttccatatccgtgtttacttaaggtgacgtcaatatttgtgtgacagtgcaatgtaataaaaatatgttaactgttcactggtttttattgctttttgggcggggactcaggtatataagtagaagcagacctgtgtggttagctcataggagctggctttcatccatggaggtttgggccattttggaagaccttaggaagactaggcaactgttagagaacgcttcggacggagtctccggtttttggagattctggttcgctagtgaattagctagggtagtttttaggataaaacaggactataaacaagaatttgaaaagttgttggtagattgcccaggactttttgaagctcttaatttgggccatcaggttcactttaaagaaaaagttttatcagttttagacttttcaaccccaggtagaactgctgctgctgtggcttttcttacttttatattagataaatggatcccgcagactcatttcagcaggggatacgttttggatttcatagccacagcattgtggagaacatggaaggttcgcaagatgaggacaatcttaggttactggccagtgcagcctttgggtgtagcgggaatcctgaggcatccaccggtcatgccagcggttctggaggaggaacagcaagaggacaacccgagagccggcctggaccctccagtggaggaggcggagtagctgacttgtctcctgaactgcaacgggtgcttactggatctacgtccactggacgggataggggcgttaagagggagagggcatctagtggtactgatgctagatctgagttggctttaagtttaatgagtcgcagacgtcctgaaaccatttggtggcatgaggttcagaaagagggaagggatgaagtttctgtattgcaggagaaatattcactggaacaggtgaaaacatgttggttggagcctgaggatgattgggaggtggccattaaaaattatgccaagatagctttgaggcctgataaacagtataagattactagacggattaatatccggaatgcttgttacatatctggaaatggggctgaggtggtaatagatactcaagacaaggcagttattagatgctgcatgatggatatgtggcctggggtagtcggtatggaagcagtaacttttgtaaatgttaagtttaggggagatggttataatggaatagtgtttatggccaataccaaacttatattgcatggttgtagcttttttggtttcaacaatacctgtgtagatgcctggggacaggttagtgtacggggatgtagtttctatgcgtgttggattgccacagctggcagaaccaagagtcaattgtctctgaagaaatgcatatttcaaagatgtaacctgggcattctgaatgaaggcgaagcaagggtccgccactgcgcttctacagatactggatgttttattttgattaagggaaatgccagcgtaaagcataacatgatttgcggtgcttccgatgagaggccttatcaaatgctcacttgtgctggtgggcattgtaatatgctggctactgtgcatattgtttcccatcaacgcaaaaaatggcctgtttttgatcacaatgtgatgacgaagtgtaccatgcatgcaggtgggcgtagaggaatgtttatgccttaccagtgtaacatgaatcatgtgaaagtgttgttggaaccagatgccttttccagaatgagcctaacaggaatttttgacatgaacatgcaaatctggaagatcctgaggtatgatgatacgagatcgagggtacgcgcatgcgaatgcggaggcaagcatgccaggttccagccggtgtgtgtagatgtgactgaagatctcagaccggatcatttggttattgcccgcactggagcagagttcggatccagtggagaagaaactgactaaggtgagtattgggaaaactttggggtgggattttcagatggacagattgagtaaaaatttgttttttctgtcttgcagctgtcatgagtggaaacgcttcttttaaggggggagtcttcagcccttatctgacagggcgtctcccatcctgggcaggagttcgtcagaatgttatgggatctactgtggatggaagacccgtccaacccgccaattcttcaacgctgacctatgctactttaagttcttcacctttggacgcagctgcagctgccgccgccgcttctgttgccgctaacactgtgcttggaatgggttactatggaagcatcatggctaattccacttcctctaataacccttctaccctgactcaggacaagttacttgtccttttggcccagctggaggctttgacccaacgtctgggtgaactttctcagcaggtggtcgagttgcgagtacaaactgagtctgctgtcggcacggcaaagtctaaataaaaaaatcccagaatcaatgaataaataaacaagcttgttgttgatttaaaatcaagtgtttttatttcatttttcgcgcacggtatgccctagaccaccgatctctatcattgagaactcggtggattttttccaggatcctatagaggtgggattgaatgtttagatacatgggcattaggccgtctttggggtggagatagctccattgaagggattcatgctccggggtagtgttgtaaatcacccagtcataacaaggtcgcagtgcatggtgttgcacaatatcttttagaagtaggctgattgccacagataagcccttggtgtaggtgtttacaaaccggttgagctgggatgggtgcattcggggtgaaattatgtgcattttggattggatttttaagttggcaatattgccgccaagatcccgtcttgggttcatgttatgaaggaccaccaagacggtgtatccggtacatttaggaaatttatcgtgcagcttggatggaaaagcgtggaaaaatttggagacacccttgtgtcctccaagattttccatgcactcatccatgataatagcaatggggccgtgggcagcggcgcgggcaaacacgttccgtgggtctgacacatcatagttatgttcctgagttaaatcatcataagccattttaatgaatttggggcggagagtaccagattggggtatgaatgttccttcgggccccggagcatagttcccctcacagatttgcatttcccaagctttcagttccgagggtggaatcatgtccacctggggggctatgaaaaacaccgtttctggggcgggggtgattaattgtgatgatagcaaatttctgagcaattgagatttgccacatccggtggggccataaatgattccgattacgggttgcaggtggtagtttagggaacggcaactgccgtcttctcgaagcaagggggccacctcgttcatcatttcccttacatgcatattttcccgcaccaaatccattaggaggcgctctcctcctagtgatagaagttcttgtagtgaggaaaagtttttcagcggtttcagaccgtcagccatgggcattttggagagagtttgctgcaaaagttctagtctgttccacagttcagtgatgtgttctatggcatctcgatccagcagacctcctcgtttcgcgggtttggacggctcctggaatagggtatgagacgatgggcgtccagcgctgccagggttcggtccttccagggtctcagtgttcgagtcagggttgtttccgtcacagtgaaggggtgtgcgcctgcttgggcgcttgccagggtgcgcttcagactcatcctgctggtcgaaaacttctgtcgcttggcgccctgtatgtcggccaagtagcagtttaccatgagttcgtagttgagcgcctcggctgcgtggcctttggcgcggagcttacctttggaagttttcttgcataccgggcagtataggcatttcagcgcatacaacttgggcgcaaggaaaacggattctggggagtatgcatctgcgccgcaggaggcgcaaacagtttcacattccaccagccaggttaaatccggttcattggggtcaaaaacaagttttccgccatattttttgatgcgtttcttacctttggtctccatgagttcgtgtcctcgttgagtgacaaacaggctgtccgtgtccccgtagactgattttacaggcctcttctccagtggagtgcctcggtcttcttcgtacaggaactctgaccactctgatacaaaggcgcgcgtccaggccagcacaaaggaggctatgtgggaggggtagcgatcgttgtcaaccagggggtccaccttttccaaagtatgcaaacacatgtcaccctcttcaacatccaggaatgtgattggcttgtaggtgtatttcacgtgacctggggtccccgctgggggggtataaaagggggcggttctttgctcttcctcactgtcttccggatcgctgtccaggaacgtcagctgttggggtaggtattccctctcgaaggcgggcatgacctctgcactcaggttgtcagtttctaagaacgaggaggatttgatattgacagtgccggttgagatgcctttcatgaggttttcgtccatttggtcagaaaacacaatttttttattgtcaagtttggtggcaaatgatccatacagggcgttggataaaagtttggcaatggatcgcatggtttggttcttttccttgtccgcgcgctctttggcggcgatgttgagttggacatactcgcgtgccaggcacttccattcggggaagatagttgttaattcatctggcacgattctcacttgccaccctcgattatgcaaggtaattaaatccacactggtggccacctcgcctcgaaggggttcattggtccaacagagcctacctcctttcctagaacagaaagggggaagtgggtctagcataagttcatcgggagggtctgcatccatggtaaagattcccggaagtaaatccttatcaaaatagctgatgggagtggggtcatctaaggccatttgccattctcgagctgccagtgcgcgctcatatgggttaaggggactgccccatggcatgggatgggtgagtgcagaggcatacatgccacagatgtcatagacgtagatgggatcctcaaagatgcctatgtaggttggatagcatcgcccccctctgatacttgctcgcacatagtcatatagttcatgtgatggcgctagcagccccggacccaagttggtgcgattgggtttttctgttctgtagacgatctggcgaaagatggcgtgagaattggaagagatggtgggtctttgaaaaatgttgaaatgggcatgaggtagacctacagagtctctgacaaagtgggcataagattcttgaagcttggttaccagttcggcggtgacaagtacgtctagggcgcagtagtcaagtgtttcttgaatgatgtcataacctggttggtttttcttttcccacagttcgcggttgagaaggtattcttcgcgatccttccagtactcttctagcggaaacccgtctttgtctgcacggtaagatcctagcatgtagaactgattaactgccttgtaagggcagcagcccttctctacgggtagagagtatgcttgagcagcttttcgtagcgaagcgtgagtaagggcaaaggtgtctctgaccatgactttgagaaattggtatttgaagtcgatgtcgtcacaggctccctgttcccagagttggaagtctacccgtttcttgtaggcggggttgggcaaagcgaaagtaacatcattgaagagaatcttaccggctctgggcataaaattgcgagtgatgcgaaaaggctgtggtacttccgctcgattgttgatcacctgggcagctaggacgatctcgtcgaaaccgttgatgttgtgtcctacgatgtataattctatgaaacgcggcgtgcctctgacgtgaggtagcttactgagctcatcaaaggttaggtctgtggggtcagataaggcgtagtgttcgagagcccattcgtgcaggtgaggatttgcatgtaggaatgatgaccaaagatctaccgccagtgctgtttgtaactggtcccgatactgacgaaaatgccggccaattgccattttttctggagtgacacagtagaaggttctggggtcttgttgccatcgatcccacttgagtttaatggctagatcgtgggccatgttgacgagacgctcttctcctgagagtttcatgaccagcatgaaaggaactagttgtttgccaaaggatcccatccaggtgtaagtttccacatcgtaggtcaggaagagtctttctgtgcgaggatgagagccgatcgggaagaactggatttcctgccaccagttggaggattggctgttgatgtgatggaagtagaagtttctgcggcgcgccgagcattcgtgtttgtgcttgtacagacggccgcagtagtcgcagcgttgcacgggttgtatctcgtgaatgagttgtacctggcttcccttgacgagaaatttcagtgggaagccgaggcctggcgattgtatctcgtgctcttctatattcgctgtatcggcctgttcatcttctgtttcgatggtggtcatgctgacgagcccccgcgggaggcaagtccagacctcggcgcgggaggggcggagctgaaggacgagagcgcgcaggctggagctgtccagagtcctgagacgctgcggactcaggttagtaggtagggacagaagattaacttgcatgatcttttccagggcgtgcgggaggttcagatggtacttgatttccacaggttcgtttgtagagacgtcaatggcttgcagggttccgtgtcctttgggcgccactaccgtacctttgttttttcttttgatcggtggtggctctcttgcttcttgcatgctcagaagcggtgacggggacgcgcgccgggcggcagcggttgttccggacccgagggcatggctggtagtggcacgtcggcgccgcgcacgggcaggttctggtactgcgctctgagaagacttgcgtgcgccaccacgcgtcgattgacgtcttgtatctgacgtctctgggtgaaagctaccggccccgtgagcttgaacctgaaagagagttcaacagaatcaatttcggtatcgttaacggcagcttgtctcagtatttcttgtacgtcaccagagttgtcctggtaggcgatctccgccatgaactgctcgatttcttcctcctgaagatctccgcgacccgctctttcgacggtggccgcgaggtcattggagatacggcccatgagttgggagaatgcattcatgcccgcctcgttccagacgcggctgtaaaccacggccccctcggagtctcttgcgcgcatcaccacctgagcgaggttaagctccacgtgtctggtgaagaccgcatagttgcataggcgctgaaaaaggtagttgagtgtggtggcaatgtgttcggcgacgaagaaatacatgatccatcgtctcagcggcatttcgctaacatcgcccagagcttccaagcgctccatggcctcgtagaagtccacggcaaaattaaaaaactgggagtttcgcgcggacacggtcaattcctcctcgagaagacggatgagttcggctatggtggcccgtacttcgcgttcgaaggctcccgggatctcttcttcctcttctatctcttcttccactaacatctcttcttcgtcttcaggcgggggcggagggggcacgcggcgacgtcgacggcgcacgggcaaacggtcgatgaatcgttcaatgacctctccgcggcggcggcgcatggtttcagtgacggcgcggccgttctcgcgcggtcgcagagtaaaaacaccgccgcgcatctccttaaagtggtgactgggaggttctccgtttgggagggagagggcgctgattatacattttattaattggcccgtagggactgcacgcagagatctgatcgtgtcaagatccacgggatctgaaaacctttcgacgaaagcgtctaaccagtcacagtcacaaggtaggctgagtacggcttcttgtgggcgggggtggttatgtgttcggtctgggtcttctgtttcttcttcatctcgggaaggtgagacgatgctgctggtgatgaaattaaagtaggcagttctaagacggcggatggtggcgaggagcaccaggtctttgggtccggcttgctggatacgcaggcgattggccattccccaagcattatcctgacatctagcaagatctttgtagtagtcttgcatgagccgttctacgggcacttcttcctcacccgttctgccatgcatacgtgtgagtccaaatccgcgcattggttgtaccagtgccaagtcagctacgactctttcggcgaggatggcttgctgtacttgggtaagggtggcttgaaagtcatcaaaatccacaaagcggtggtaagctcctgtattaatggtgtaagcacagttggccatgactgaccagttaactgtctggtgaccagggcgcacgagctcggtgtatttaaggcgcgaataggcgcgggtgtcaaagatgtaatcgttgcaggtgcgcaccagatactggtaccctataagaaaatgcggcggtggttggcggtagagaggccatcgttctgtagctggagcgccaggggcgaggtcttccaacataaggcggtgatagccgtagatgtacctggacatccaggtgattcctgcggcggtagtagaagcccgaggaaactcgcgtacgcggttccaaatgttgcgtagcggcatgaagtagttcattgtaggcacggtttgaccagtgaggcgcgcgcagtcattgatgctctatagacacggagaaaatgaaagcgttcagcgactcgactccgtagcctggaggaacgtgaacgggttgggtcgcggtgtaccccggttcgagacttgtactcgagccggccggagccgcggctaacgtggtattggcactcccgtctcgacccagcctacaaaaatccaggatacggaatcgagtcgttttgctggtttccgaatggcagggaagtgagtcctatttttttttttttgccgctcagatgcatcccgtgctgcgacagatgcgcccccaacaacagcccccctcgcagcagcagcagcagcaatcacaaaaggctgtccctgcaactactgcaactgccgccgtgagcggtgcgggacagcccgcctatgatctggacttggaagagggcgaaggactggcacgtctaggtgcgccttcacccgagcggcatccgcgagttcaactgaaaaaagattctcgcgaggcgtatgtgccccaacagaacctatttagagacagaagcggcgaggagccggaggagatgcgagcttcccgctttaacgcgggtcgtgagctgcgtcacggtttggaccgaagacgagtgttgcgggacgaggatttcgaagttgatgaaatgacagggatcagtcctgccagggcacacgtggctgcagccaaccttgtatcggcttacgagcagacagtaaaggaagagcgtaacttccaaaagtcttttaataatcatgtgcgaaccctgattgcccgcgaagaagttacccttggtttgatgcatttgtgggatttgatggaagctatcattcagaaccctactagcaaacctctgaccgcccagctgtttctggtggtgcaacacagcagagacaatgaggctttcagagaggcgctgctgaacatcaccgaacccgaggggagatggttgtatgatcttatcaacattctacagagtatcatagtgcaggagcggagcctgggcctggccgagaaggtggctgccatcaattactcggttttgagcttgggaaaatattacgctcgcaaaatctacaagactccatacgttcccatagacaaggaggtgaagatagatgggttctacatgcgcatgacgctcaaggtcttgaccctgagcgatgatcttggggtgtatcgcaatgacagaatgcatcgcgcggttagcgccagcaggaggcgcgagttaagcgacagggaactgatgcacagtttgcaaagagctctgactggagctggaaccgagggtgagaattacttcgacatgggagctgacttgcagtggcagcctagtcgcagggctctgagcgccgcgacggcaggatgtgagcttccttacatagaagaggcggatgaaggcgaggaggaagagggcgagtacttggaagactgatggcacaacccgtgttttttgctagatggaacagcaagcaccggatcccgcaatgcgggcggcgctgcagagccagccgtccggcattaactcctcggacgattggacccaggccatgcaacgtatcatggcgttgacgactcgcaaccccgaagcctttagacagcaaccccaggccaaccgtctatcggccatcatggaagctgtagtgccttcccgctctaatcccactcatgagaaggtcctggccatcgtgaacgcgttggtggagaacaaagctattcgtccagatgaggccggactggtatacaacgctctcttagaacgcgtggctcgctacaacagtagcaatgtgcaaaccaatttggaccgtatgataacagatgtacgcgaagccgtgtctcagcgcgaaaggttccagcgtgatgccaacctgggttcgctggtggcgttaaatgctttcttgagtactcagcctgctaatgtgccgcgtggtcaacaggattatactaactttttaagtgctttgagactgatggtatcagaagtacctcagagcgaagtgtatcagtccggtcctgattacttctttcagactagcagacagggcttgcagacggtaaatctgagccaagcttttaaaaaccttaaaggtttgtggggagtgcatgccccggtaggagaaagagcaaccgtgtctagcttgttaactccgaactcccgcctattattactgttggtagctcctttcaccgacagcggtagcatcgaccgtaattcctatttgggttacctactaaacctgtatcgcgaagccatagggcaaagtcaggtggacgagcagacctatcaagaaattacccaagtcagtcgcgctttgggacaggaagacactggcagtttggaagccactctgaacttcttgcttaccaatcggtctcaaaagatccctcctcaatatgctcttactgcggaggaggagaggatccttagatatgtgcagcagagcgtgggattgtttctgatgcaagagggggcaactccgactgcagcactggacatgacagcgcgaaatatggagcccagcatgtatgccagtaaccgacctttcattaacaaactgctggactacttgcacagagctgccgctatgaactctgattatttcaccaatgccatcttaaacccgcactggctgcccccacctggtttctacacgggcgaatatgacatgcccgaccctaatgacggatttctgtgggacgacgtggacagcgatgttttttcacctctttctgatcatcgcacgtggaaaaaggaaggcggcgatagaatgcattcttctgcatcgctgtccggggtcatgggtgctaccgcggctgagcccgagtctgcaagtccttttcctagtctacccttttctctacacagtgtacgtagcagcgaagtgggtagaataagtcgcccgagtttaatgggcgaagaggagtatctaaacgattccttgctcagaccggcaagagaaaaaaatttcccaaacaatggaatagaaagtttggtggataaaatgagtagatggaagacttatgctcaggatcacagagacgagcctgggatcatggggattacaagtagagcgagccgtagacgccagcgccatgacagacagaggggtcttgtgtgggacgatgaggattcggccgatgatagcagcgtgctggacttgggtgggagaggaaggggcaacccgtttgctcatttgcgccctcgcttgggtggtatgttgtaaaaaaaaataaaaaaaaaactcaccaaggccatggcgacgagcgtacgttcgttcttctttattatctgtgtctagtataatgaggcgagtcgtgctaggcggagcggtggtgtatccggagggtcctcctccttcgtacgagagcgtgatgcagcagcagcaggcgacggcggtgatgcaatccccactggaggctccctttgtgcctccgcgatacctggcacctacggagggcagaaacagcattcgttattcggaactggcacctcagtacgataccaccaggttgtatctggtggacaacaagtcggcggacattgcttctctgaactatcagaatgaccacagcaacttcttgaccacggtggtgcaaaacaatgactttacccctacggaagccagcacccagaccattaactttgatgaacgatcgcggtggggcggtcagctaaagaccatcatgcatactaacatgccaaacgtgaacgagtatatgtttagtaacaagttcaaagcgcgtgtgatggtgtccagaaaacctcccgacggtgctgcagttggggatacttatgatcacaagcaggatattttgaaatatgagtggttcgagtttactttgccagaaggcaacttttcagttactatgactattgatttgatgaacaatgccatcatagataattacttgaaagtgggtagacagaatggagtgcttgaaagtgacattggtgttaagttcgacaccaggaacttcaagctgggatgggatcccgaaaccaagttgatcatgcctggagtgtatacgtatgaagccttccatcctgacattgtcttactgcctggctgcggagtggattttaccgagagtcgtttgagcaaccttcttggtatcagaaaaaaacagccatttcaagagggttttaagattttgtatgaagatttagaaggtggtaatattccggccctcttggatgtagatgcctatgagaacagtaagaaagaacaaaaagccaaaatagaagctgctacagctgctgcagaagctaaggcaaacatagttgccagcgactctacaagggttgctaacgctggagaggtcagaggagacaattttgcgccaacacctgttccgactgcagaatcattattggccgatgtgtctgaaggaacggacgtgaaactcactattcaacctgtagaaaaagatagtaagaatagaagctataatgtgttggaagacaaaatcaacacagcctatcgcagttggtatctttcgtacaattatggcgatcccgaaaaaggagtgcgttcctggacattgctcaccacctcagatgtcacctgcggagcagagcaggtctactggtcgcttccagacatgatgaaggatcctgtcactttccgctccactagacaagtcagtaactaccctgtggtgggtgcagagcttatgcccgtcttctcaaagagcttctacaacgaacaagctgtgtactcccagcagctccgccagtccacctcgcttacgcacgtcttcaaccgctttcctgagaaccagattttaatccgtccgccggcgcccaccattaccaccgtcagtgaaaacgttcctgctctcacagatcacgggaccctgccgttgcgcagcagtatccggggagtccaacgtgtgaccgttactgacgccagacgccgcacctgtccctacgtgtacaaggcactgggcatagtcgcaccgcgcgtcctttcaagccgcactttctaaaaaaaaaaaaaatgtccattcttatctcgcccagtaataacaccggttggggtctgcgcgctccaagcaagatgtacggaggcgcacgcaaacgttctacccaacatcctgtccgtgttcgcggacattttcgcgctccatggggcgccctcaagggccgcactcgcgttcgaaccaccgtcgatgatgtaatcgatcaggtggttgccgacgcccgtaattatactcctactgcgcctacatctactgtggatgcagttattgacagtgtagtggctgacgctcgcaactatgctcgacgtaagagccggcgaaggcgcattgccagacgccaccgagctaccactgccatgcgagccgcaagagctctgctacgaagagctagacgcgtggggcgaagagccatgcttagggcggccagacgtgcagcttcgggcgccagcgccggcaggtcccgcaggcaagcagccgctttcgcagcggcgactattgccgacatggcccaatcgcgaagaggcaatgtatactgggtgcgtgacgctgccaccggtcaacgtgtacccgtgcgcacccgtccccctcgcacttagaagatactgagcagtctccgatgttgtgtcccagcggcgaggatgtccaagcgcaaatacaaggaagaaatgctgcaggttatcgcacctgaagtctacggccaaccgttgaaggatgaaaaaaaaccccgcaaaatcaagcgggttaaaaaggacaaaaaagaagaggaagatggcgatgatgggctggcggagtttgtgcgcgagtttgccccacggcgacgcgtgcaatggcgtgggcgcaaagttcgacatgtgttgagacctggaacttcggtggtctttacacccggcgagcgttcaagcgctacttttaagcgttcctatgatgaggtgtacggggatgatgatattcttgagcaggcggctgaccgattaggcgagtttgcttatggcaagcgtagtagaataacttccaaggatgagacagtgtcgatacccttggatcatggaaatcccacccctagtcttaaaccggtcactttgcagcaagtgttacccgtaactccgcgaacaggtgttaaacgcgaaggtgaagatttgtatcccactatgcaactgatggtacccaaacgccagaagttggaggacgttttggagaaagtaaaagtggatccagatattcaacctgaggttaaagtgagacccattaagcaggtagcgcctggtctgggggtacaaactgtagacattaagattcccactgaaagtatggaagtgcaaactgaacccgcaaagcctactgccacctccactgaagtgcaaacggatccatggatgcccatgcctattacaactgacgccgccggtcccactcgaagatcccgacgaaagtacggtccagcaagtctgttgatgcccaattatgttgtacacccatctattattcctactcctggttaccgaggcactcgctactatcgcagccgaaacagtacctcccgccgtcgccgcaagacacctgcaaatcgcagtcgtcgccgtagacgcacaagcaaaccgactcccggcgccctggtgcggcaagtgtaccgcaatggtagtgcggaacctttgacactgccgcgtgcgcgttaccatccgagtatcatcacttaatcaatgttgccgctgcctccttgcagatatggccctcacttgtcgccttcgcgttcccatcactggttaccgaggaagaaactcgcgccgtagaagagggatgttgggacgcggaatgcgacgctacaggcgacggcgtgctatccgcaagcaattgcggggtggttttttaccagccttaattccaattatcgctgctgcaattggcgcgataccaggcatagcttccgtggcggttcaggcctcgcaacgacattgacattggaaaaaaacgtataaataaaaaaaaaaaaatacaatggactctgacactcctggtcctgtgactatgttttcttagagatggaagacatcaatttttcatccttggctccgcgacacggcacgaagccgtacatgggcacctggagcgacatcggcacgagccaactgaacgggggcgccttcaattggagcagtatctggagcgggcttaaaaattttggctcaaccataaaaacatacgggaacaaagcttggaacagcagtacaggacaggcgcttagaaataaacttaaagaccagaacttccaacaaaaagtagtcgatgggatagcttccggcatcaatggagtggtagatttggctaaccaggctgtgcagaaaaagataaacagtcgtttggacccgccgccagcaaccccaggtgaaatgcaagtggaggaagaaattcctccgccagaaaaacgaggcgacaagcgtccgcgtcccgatttggaagagacgctggtgacgcgcgtagatgaaccgccttcttatgaggaagcaacgaagcttggaatgcccaccactagaccgatagccccaatggccaccggggtgatgaaaccttctcagttgcatcgacccgtcaccttggatttgccccctccccctgctgctactgctgtacccgcttctaagcctgtcgctgccccgaaaccagtcgccgtagccaggtcacgtcccgggggcgctcctcgtccaaatgcgcactggcaaaatactctgaacagcatcgtgggtctaggcgtgcaaagtgtaaaacgccgtcgctgcttttaattaaatatggagtagcgcttaacttgcctatctgtgtatatgtgtcattacacgccgtcacagcagcagaggaaaaaaggaagaggtcgtgcgtcgacgctgagttactttcaagatggccaccccatcgatgctgccccaatgggcatacatgcacatcgccggacaggatgcttcggagtacctgagtccgggtctggtgcagttcgcccgcgccacagacacctacttcaatctgggaaataagtttagaaatcccaccgtagcgccgacccacgatgtgaccaccgaccgtagccagcggctcatgttgcgcttcgtgcccgttgaccgggaggacaatacatactcttacaaagtgcggtacaccctggccgtgggcgacaacagagtgctggatatggccagcacgttctttgacattaggggtgtgttggacagaggtcccagtttcaaaccctattctggtacggcttacaactccctggctcctaaaggcgctccaaatacatctcagtggattgcagaaggtgtaaaaaatacaactggtgaggaacacgtaacagaagaggaaaccaatactactacttacacttttggcaatgctcctgtaaaagctgaagctgaaattacaaaagaaggactcccagtaggtttggaagtttcagatgaagaaagtaaaccgatttatgctgataaaacatatcagccagaacctcagctgggagatgaaacttggactgaccttgatggaaaaaccgaaaagtatggaggcagggctctcaaacccgatactaagatgaaaccatgctacgggtcctttgccaaacctactaatgtgaaaggcggtcaggcaaaacaaaaaacaacggagcagccaaatcagaaagtcgaatatgatatcgacatggagttttttgatgcggcatcgcagaaaacaaacttaagtcctaaaattgtcatgtatgcagaaaatgtaaatttggaaactccagacactcatgtagtgtacaaacctggaacagaagacacaagttccgaagctaatttgggacaacaatctatgcccaacagacccaactacattggcttcagagataactttattggacttatgtactataacagtactggtaacatgggggtgctggctggtcaagcgtctcagttaaatgcagtggttgacttgcaggacagaaacacagaactttcttaccaactcttgcttgactctctgggcgacagaaccagatactttagcatgtggaatcaggctgtggacagttatgatcctgatgtacgtgttattgaaaatcatggtgtggaagatgaacttcccaactactgttttccactggacggcataggtgttccaacaaccagttacaaatcaatagttccaaatggagacaatgcgcctaattggaaggaacctgaagtaaatggaacaagtgagatcggacagggtaatttgtttgccatggaaattaaccttcaagccaatctatggcgaagtttcctttattccaatgtggctctatatctcccagactcgtacaaatacaccccgtccaatgtcactcttccagaaaacaaaaacacctacgactacatgaacgggcgggtggtgccgccatctctagtagacacctatgtgaacattggtgccaggtggtctctggatgccatggacaatgtcaacccattcaaccaccaccgtaacgctggcttgcgttaccgatccatgcttctgggtaacggacgttatgtgcctttccacatacaagtgcctcaaaaattcttcgctgttaaaaacctgctgcttctcccaggctcctacacttatgagtggaactttaggaaggatgtgaacatggttctacagagttccctcggtaacgacctgcgggtagatggcgccagcatcagtttcacgagcatcaacctctatgctacttttttccccatggctcacaacaccgcttccacccttgaagccatgctgcggaatgacaccaatgatcagtcattcaacgactacctatctgcagctaacatgctctaccccattcctgccaatgcaaccaatattcccatttccattccttctcgcaactgggcggctttcagaggctggtcatttaccagactgaaaaccaaagaaactccctctttggggtctggatttgacccctactttgtctattctggttctattccctacctggatggtaccttctacctgaaccacacttttaagaaggtttccatcatgtttgactcttcagtgagctggcctggaaatgacaggttactatctcctaacgaatttgaaataaagcgcactgtggatggcgaaggctacaacgtagcccaatgcaacatgaccaaagactggttcttggtacagatgctcgccaactacaacatcggctatcagggcttctacattccagaaggatacaaagatcgcatgtattcatttttcagaaacttccagcccatgagcaggcaggtggttgatgaggtcaattacaaagacttcaaggccgtcgccataccctaccaacacaacaactctggctttgtgggttacatggctccgaccatgcgccaaggtcaaccctatcccgctaactatccctatccactcattggaacaactgccgtaaatagtgttacgcagaaaaagttcttgtgtgacagaaccatgtggcgcataccgttctcgagcaacttcatgtctatgggggcccttacagacttgggacagaatatgctctatgccaactcagctcatgctctggacatgacctttgaggtggatcccatggatgagcccaccctgctttatcttctcttcgaagttttcgacgtggtcagagtgcatcagccacaccgcggcatcatcgaggcagtctacctgcgtacaccgttctcggccggtaacgctaccacgtaagaagcttcttgcttcttgcaaatagcagctgcaaccatggcctgcggatcccaaaacggctccagcgagcaagagctcagagccattgtccaagacctgggttgcggaccctattttttgggaacctacgataagcgcttcccggggttcatggcccccgataagctcgcctgtgccattgtaaatacggccggacgtgagacggggggagagcactggttggctttcggttggaacccacgttctaacacctgctacctttttgatccttttggattctcggatgatcgtctcaaacagatttaccagtttgaatatgagggtctcctgcgccgcagcgctcttgctaccaaggaccgctgtattacgctggaaaaatctacccagaccgtgcagggtccccgttctgccgcctgcggacttttctgctgcatgttccttcacgcctttgtgcactggcctgaccgtcccatggacggaaaccccaccatgaaattgctaactggagtgccaaacaacatgcttcattctcctaaagtccagcccaccctgtgtgacaatcaaaaagcactctaccattttcttaatacccattcgccttattttcgctcccatcgtacacacatcgaaagggccactgcgttcgaccgtatggatgttcaataatgactcatgtaaacaacgtgttcaataaacatcactttatttttttacatgtatcaaggctctgcattacttatttatttacaagtcgaatgggttctgacgagaatcagaatgacccgcaggcagtgatacgttgcggaactgatacttgggttgccacttgaattcgggaatcaccaacttgggaaccggtatatcgggcaggatgtcactccacagctttctggtcagctgcaaagctccaagcaggtcaggagccgaaatcttgaaatcacaattaggaccagtgctttgagcgcgagagttgcggtacaccggattgcagcactgaaacaccatcagcgacggatgtctcacgcttgccagcacggtgggatctgcaatcatgcccacatccagatcttcagcattggcaatgctgaacggggtcatcttgcaggtctgcctacccatggcgggcacccaattaggcttgtggttgcaatcgcagtgcagggggatcagtatcatcttggcctgatcctgtctgattcctggatacacggctctcatgaaagcatcatattgcttgaaagcctgctgggctttactaccctcggtataaaacatcccgcaggacctgctcgaaaactggttagctgcacagccggcatcattcacacagcagcgggcgtcattgttagctatttgcaccacacttctgccccagcggttttgggtgattttggttcgctcgggattctcctttaaggctcgttgtccgttctcgctggccacatccatctcgataatctgctccttctgaatcataatattgccatgcaggcacttcagcttgccctcataatcattgcagccatgaggccacaacgcacagcctgtacattcccaattatggtgggcgatctgagaaaaagaatgtatcattccctgcagaaatcttcccatcatcgtgctcagtgtcttgtgactagtgaaagttaactggatgcctcggtgctcctcgtttacgtactggtgacagatgcgcttgtattgttcgtgttgctcaggcattagtttaaaagaggttctaagttcgttatccagcctgtacttctccatcagcagacacatcacttccatgcctttctcccaagcagacaccaggggcaagctaatcggattcttaacagtgcaggcagcagctcctttagccagagggtcatctttagcgatcttctcaatgcttcttttgccatccttctcaacgatgcgcacgggcgggtagctgaaacccactgctacaagttgcgcctcttctctttcttcttcgctgtcttgactgatgtcttgcatggggatatgtttggtcttccttggcttctttttggggggtatcggaggaggaggactgtcgctccgttccggagacagggaggattgtgacgtttcgctcaccattaccaactgactgtcggtagaagaacctgaccccacacggcgacaggtgtttctcttcgggggcagaggtggaggcgattgcgaagggctgcggtccgacctggaaggcggatgactggcagaaccccttccgcgttcgggggtgtgctccctgtggcggtcgcttaactgatttccttcgcggctggccattgtgttctcctaggcagagaaacaacagacatggaaactcagccattgctgtcaacatcgccacgagtgccatcacatctcgtcctcagcgacgaggaaaaggagcagagcttaagcattccaccgcccagtcctgccaccacctctaccctagaagataaggaggtcgacgcatctcatgacatgcagaataaaaaagcgaaagagtctgagacagacatcgagcaagacccgggctatgtgacaccggtggaacacgaggaagagttgaaacgctttctagagagagaggatgaaaactgcccaaaacaacgagcagataactatcaccaagatgctggaaatagggatcagaacaccgactacctcatagggcttgacggggaagacgcgctccttaaacatctagcaagacagtcgctcatagtcaaggatgcattattggacagaactgaagtgcccatcagtgtggaagagctcagccgcgcctacgagcttaacctcttttcacctcgtactccccccaaacgtcagccaaacggcacctgcgagccaaatcctcgcttaaacttttatccagcttttgctgtgccagaagtactggctacctatcacatcttttttaaaaatcaaaaaattccagtctcctgccgcgctaatcgcacccgcgccgatgccctactcaatctgggacctggttcacgcttacctgatatagcttccttggaagaggttccaaagatcttcgagggtctgggcaataatgagactcgggccgcaaatgctctgcaaaagggagaaaatggcatggatgagcatcacagcgttctggtggaattggaaggcgataatgccagactcgcagtactcaagcgaagcatcgaggtcacacacttcgcatatcccgctgtcaacctgccccctaaagtcatgacggcggtcatggaccagttactcattaagcgcgcaagtcccctttcagaagacatgcatgacccagatgcctgtgatgagggtaaaccagtggtcagtgatgagcagctaacccgatggctgggcaccgactctcccagggatttggaagagcgtcgcaagcttatgatggccgtggtgctggttaccgtagaactagagtgtctccgacgtttctttaccgattcagaaaccttgcgcaaactcgaagagaatctgcactacacttttagacacggctttgtgcggcaggcatgcaagatatctaacgtggaactcaccaacctggtttcctacatgggtattctgcatgagaatcgcctaggacaaagcgtgctgcacagcaccctgaagggggaagcccgccgtgattacatccgcgattgtgtctatctgtacctgtgccaaacgtggcaaaccggcatgggtgtatggcagcaatgtttagaagaacagaacttgaaagagcttgacaagctcttacagaaatctcttaaggttctgtggacagggttcgacgagcgcaccgtcgcttccgacctggcagacctcatcttcccagagcgtctcagggttactttgcgaaacggattgcctgactttatgagccagagcatgcttaacaattttcgctctttcatcctggaacgctccggtatcctgcccgccacctgctgcgcactgccctccgactttgtgcctctcacctaccgcgagtgccccccgccgctatggagtcactgctacctgttccgtctggccaactatctctcctaccactcggatgtgatcgaggatgtgagcggagacggcttgctggagtgtcactgccgctgcaatctgtgcacgccccaccggtccctagcttgcaacccccagttgatgagcgaaacccagataataggcacctttgaattgcaaggccccagcagccaaggcgatgggtcttctcctgggcaaagtttaaaactgaccccgggactgtggacctccgcctacttgcgcaagtttgctccggaagattaccacccctatgaaatcaagttctatgaggaccaatcacagcctccaaaggccgaactttcggcctgcgtcatcacccagggggcaattctggcccaattgcaagccatccaaaaatcccgccaagaatttctactgaaaaagggtaagggggtctaccttgacccccagaccggcgaggaactcaacacaaggttccctcaggatgtcccaacgacgagaaaacaagaagttgaaggtgcagccgccgcccccagaagatatggaggaagattgggacagtcaggcagaggaggcggaggaggacagtctggaggacagtctggaggaagacagtttggaggaggaaaacgaggaggcagaggaggtggaagaagtaaccgccgacaaacagttatcctcggctgcggagacaagcaacagcgctaccatctccgctccgagtcgaggaacccggcggcgtcccagcagtagatgggacgagaccggacgcttcccgaacccaaccagcgcttccaagaccggtaagaaggatcggcagggatacaagtcctggcgggggcataagaatgccatcatctcctgcttgcatgagtgcgggggcaacatatccttcacgcggcgctacttgctattccaccatggggtgaactttccgcgcaatgttttgcattactaccgtcacctccacagcccctactatagccagcaaatcccggcagtctcgacagataaagacagcggcggcgacctccaacagaaaaccagcagcggcagttagaaaatacacaacaagtgcagcaacaggaggattaaagattacagccaacgagccagcgcaaacccgagagttaagaaatcggatctttccaaccctgtatgccatcttccagcagagtcggggtcaagagcaggaactgaaaataaaaaaccgatctctgcgttcgctcaccagaagttgtttgtatcacaagagcgaagatcaacttcagcgcactctcgaggacgccgaggctctcttcaacaagtactgcgcgctgactcttaaagagtaggcagcgaccgcgcttattcaaaaaaggcgggaattacatcatcctcgacatgagtaaagaaattcccacgccttacatgtggagttatcaaccccaaatgggattggcggcaggcgcctcccaggactactccacccgcatgaattggctcagcgccgggccttctatgatttctcgagttaatgatatacgcgcctaccgaaaccaaatacttttggaacagtcagctcttaccaccacgccccgccaacaccttaatcccagaaattggcccgccgccctagtgtaccaggaaagtcccgctcccaccactgtattacttcctcgagacgcccaggccgaagtccaaatgactaatgcaggtgcgcagttagctggcggctccaccctatgtcgtcacaggcctcggcataatataaaacgcctgatgatcagaggccgaggtatccagctcaacgacgagtcggtgagctctccgcttggtctacgaccagacggaatctttcagattgccggctgcgggagatcttccttcacccctcgtcaggctgttctgactttggaaagttcgtcttcgcaaccccgctcgggcggaatcgggaccgttcaatttgtggaggagtttactccctctgtctacttcaaccccttctccggatctcctgggcattacccggacgagttcataccgaacttcgacgcgattagcgagtcagtggacggctacgattgatgtctggtgacgcggctgagctatctcggctgcgacatctagaccactgccgccgctttcgctgctttgcccgggaactcattgagttcatctacttcgaactccccaaggatcaccctcaaggtccggcccacggagtgcggatttctatcgaaggcaaaatagactctcgcctgcaacgaattttctcccagcggcccgtgctgatcgagcgagaccagggaaacaccacggtttccatctactgcatttgtaatcaccccggattgcatgaaagcctttgctgtcttatgtgtactgagtttaataaaaactgaattaagactctcctacggactgccgcttcttcaacccggattttacaaccagaagaacgaaacttttcctgtcgtccaggactctgttaacttcacctttcctactcacaaactagaagctcaacgactacaccgcttttccagaagcattttccctactaatactactttcaaaaccggaggtgagctccaaggtcttcctacagaaaacccttgggtggaagcgggccttgtagtgctaggaattcttgcgggtgggcttgtgattattctttgctacctatacacaccttgcttcactttcttagtggtgttgtggtattggtttaaaaaatggggcccatactagtcttgcttgttttactttcgcttttggaaccgggttctgccaattacgatccatgtctagacttcgacccagaaaactgcacacttacttttgcacccgacacaagccgcatctgtggagttcttattaagtgcggatgggaatgcaggtccgttgaaattacacacaataacaaaacctggaacaataccttatccaccacatgggagccaggagttcccgagtggtacactgtctctgtccgaggtcctgacggttccatccgcattagtaacaacactttcattttttctgaaatgtgcgatctggccatgttcatgagcaaacagtattctctatggcctcctagcaaggacaacatcgtaacgttctccattgcttattgcttgtgcgcttgccttcttactgctttactgtgcgtatgcatacacctgcttgtaaccactcgcatcaaaaacgccaataacaaagaaaaaatgccttaacctctttctgtttacagacatggcttctcttacatctctcatatttgtcagcattgtcactgccgctcatggacaaacagtcgtctctatccctctaggacataattacactctcataggacccccaatcacttcagaggtcatctgggccaaactgggaagcgttgattactttgatataatctgcaacaaaacaaaaccaataatagtaacttgcaacatacaaaatcttacattgattaatgttagcaaagtttacagcggttactattatggttatgacagatacagtagtcaatatagaaattacttggttcgtgttacccagttgaaaaccacgaaaatgccaaatatggcaaagattcgatccgatgacaattctctagaaacttttacatctcccaccacacccgacgaaaaaaacatcccagattcaatgattgcaattgttgcagcggtggcagtggtgatggcactaataataatatgcatgcttttatatgcttgtcgctacaaaaagtttcatcctaaaaaacaagatctcctactaaggcttaacatttaatttctttttatacagccatggtttccactaccacattccttatgcttactagtctcgcaactctgacttctgctcgctcacacctcactgtaactataggctcaaactgcacactaaaaggacctcaaggtggtcatgtcttttggtggagaatatatgacaatggatggtttacaaaaccatgtgaccaacctggtagatttttctgcaacggcagagacctaaccattatcaacgtgacagcaaatgacaaaggcttctattatggaaccgactataaaagtagtttagattataacattattgtactgccatctaccactccagcaccccgcacaactactttctctagcagcagtgtcgctaacaatacaatttccaatccaacctttgccgcgcttttaaaacgcactgtgaataattctacaacttcacatacaacaatttccacttcaacaatcagcattatcgctgcagtgacaattggaatatctattcttgtttttaccataacctactacgcctgctgctatagaaaagacaaacataaaggtgatccattacttagatttgatatttaatttgttctttttttttttatttacagtatggtgaacaccaatcatggtacctagaaatttcttcttcaccatactcatttgtgcatttaatgtttgcgctactttcacagcagtagccacagcaaccccagactgtataggagcatttgcttcctatgcactttttgcttttgttacttgcatctgcgtatgtagcatagtctgcctggttattaattttttccaacttatagactggatccttgtgcgaattgcctacctgcgccaccatcccgaataccgcaaccaaaatatcgcggcacttcttagactcatctaaaaccatgcaggctatactaccaatatttttgcttctattgcttccctacgctgtctcaaccccagctgcctatagtactccaccagaacaccttagaaaatgcaaattccaacaaccgtggtcatttcttgcttgctatcgagaaaaatcagaaattcccccaaatttaataatgattgctggaataattaatataatctgttgcaccataatttcatttttgatataccccctatttgattttggctggaatgctcccaatgcacatgatcatccacaagacccagaggaacacattcccctacaaaacatgcaacatccaatagcgctaatagattacgaaagtgaaccacaacccccactactccctgctattagttacttcaacctaaccggcggagatgactgaaacactcaccacctccaattccgccgaggatctgctcgatatggacggccgcgtctcagaacagcgactcgcccaactacgcatccgccagcagcaggaacgcgcggccaaagagctcagagatgtcatccaaattcaccaatgcaaaaaaggcatattctgtttggtaaaacaagccaagatatcctacgagatcaccgctactgaccatcgcctctcttacgaacttggcccccaacgacaaaaatttacctgcatggtgggaatcaaccccatagttatcacccagcaaagtggagatactaagggttgcattcactgctcctgcgattccatcgagtgcacctacaccctgctgaagaccctatgcggcctaagagacctgctaccaatgaattaaaaaatgattaataaaaaatcacttacttgaaatcagcaataaggtctctgttgaaattttctcccagcagcacctcacttccctcttcccaactctggtattctaaaccccgttcagcggcatactttctccatactttaaaggggatgtcaaattttagctcctctcctgtacccacaatcttcatgtctttcttcccagatgaccaagagagtccggctcagtgactccttcaaccctgtctacccctatgaagatgaaagcacctcccaacacccctttataaacccagggtttatttccccaaatggcttcacacaaagcccaaacggagttcttactttaaaatgtttaaccccactaacaaccacaggcggatctctacagctaaaagtgggagggggacttacagtggatgacaccaacggttttttgaaagaaaacataagtgccaccacaccactcgttaagactggtcactctataggtttaccactaggagccggattgggaacgaatgaaaataaactttgtatcaaattaggacaaggacttacattcaattcaaacaacatttgcattgatgacaatattaacaccttatggacaggagtcaaccccaccgaagccaactgtcaaatcatgaactccagtgaatctaatgattgcaaattaattctaacactagttaaaactggagcactagtcactgcatttgtttatgttataggagtatctaacaattttaatatgctaactacacacagaaatataaattttactgcagagctgtttttcgattctactggtaatttactaactagactctcatccctcaaaactccacttaatcataaatcaggacaaaacatggctactggtgccattactaatgctaaaggtttcatgcccagcacgactgcctatcctttcaatgataattctagagaaaaagaaaactacatttacggaacttgttactacacagctagtgatcgcactgcttttcccattgacatatctgtcatgcttaaccgaagagcaataaatgacgagacatcatattgtattcgtataacttggtcctggaacacaggagatgccccagaggtgcaaacctctgctacaaccctagtcacctccccatttaccttttactacatcagagaagacgactgacaaataaagtttaacttgtttatttgaaaatcaattcacaaaatccgagtagttattttgcctcccccttcccatttaacagaatacaccaatctctccccacgcacagctttaaacatttggataccattagatatagacatggttttagattccacattccaaacagtttcagagcgagccaatctggggtcagtgatagataaaaatccatcgggatagtcttttaaagcgctttcacagtccaactgctgcggatggactccggagtctggatcacggtcatctggaagaagaacgatgggaatcataatccgaaaacggtatcggacgattgtgtctcatcaaacccacaagcagccgctgtctgcgtcgctccgtgcgactgctgtttatgggatcagggtccacagtgtcctgaagcatgattttaatagcccttaacatcaactttctggtgcgatgcgcgcagcaacgcattctgatttcactcaaatctttgcagtaggtacaacacattattacaatattgtttaataaaccataattaaaagcgctccagccaaaactcatatctgatataatcgcccctgcatgaccatcataccaaagtttaatataaattaaatgacgttccctcaaaaacacactacccacatacatgatctcttttggcatgtgcatattaacaatctgtctgtaccatggacaacgttggttaatcatgcaacccaatataaccttccggaaccacactgccaacaccgctcccccagccatgcattgaagtgaaccctgctgattacaatgacaatgaagaacccaattctctcgaccgtgaatcacttgagaatgaaaaatatctatagtggcacaacatagacataaatgcatgcatcttctcataatttttaactcctcaggatttagaaacatatcccagggaataggaagctcttgcagaacagtaaagctggcagaacaaggaagaccacgaacacaacttacactatgcatagtcatagtatcacaatctggcaacagcgggtggtcttcagtcatagaagctcgggtttcattttcctcacaacgtggtaactgggctctggtgtaagggtgatgtctggcgcatgatgtcgagcgtgcgcgcaaccttgtcataatggagttgcttcctgacattctcgtattttgtatagcaaaacgcggccctggcagaacacactcttcttcgccttctatcctgccgcttagcgtgttccgtgtgatagttcaagtacaaccacactcttaagttggtcaaaagaatgctggcttcagttgtaatcaaaactccatcgcatctaatcgttctgaggaaatcatccacggtagcatatgcaaatcccaaccaagcaatgcaactggattgtgtttcaagcaggagaggagagggaagagacggaagaaccatgttaatttttattccaaacgatctcgcagtacttcaaattgtagatcgcgcagatggcatctctcgcccccactgtgttggtgaaaaagcacagctagatcaaaagaaatgcgattttcaaggtgctcaacggtggcttccagcaaagcctccacgcgcacatccaagaacaaaagaataccaaaagaaggagcattttctaactcctcaatcatcatattacattcctgcaccattcccagataattttcagctttccagccttgaattattcgtgtcagttcttgtggtaaatccaatccacacattacaaacaggtcccggagggcgccctccaccaccattcttaaacacaccctcataatgacaaaatatcttgctcctgtgtcacctgtagcgaattgagaatggcaacatcaattgacatgcccttggctctaagttcttctttaagttctagttgtaaaaactctctcatattatcaccaaactgcttagccagaagccccccgggaacaagagcaggggacgctacagtgcagtacaagcgcagacctccccaattggctccagcaaaaacaagattggaataagcatattgggaaccgccagtaatatcatcgaagttgctggaaatataatcaggcagagtttcttgtaaaaattgaataaaagaaaaatttgccaaaaaaacattcaaaacctctgggatgcaaatgcaataggttaccgcgctgcgctccaacattgttagttttgaattagtctgcaaaaataaaaaaaaaaacaagcgtcatatcatagtagcctgacgaacagatggataaatcagtctttccatcacaagacaagccacagggtctccagctcgaccctcgtaaaacctgtcatcatgattaaacaacagcaccgaaagttcctcgcggtgaccagcatgaataattcttgatgaagcatacaatccagacatgttagcatcagttaacgagaaaaaacagccaacatagcctttgggtataattatgcttaatcgtaagtatagcaaagccacccctcgcggatacaaagtaaaaggcacaggagaataaaaaatataattatttctctgctgctgttcaggcaacgtcgcccccggtccctctaaatacacatacaaagcctcatcagccatggcttaccagacaaagtacagcgggcacacaaagcacaagctctaaagtgactctccaacctctccacaatatatatatacacaagccctaaactgacgtaatgggagtaaagtgtaaaaaatcccgccaaacccaacacacaccccgaaactgcgtcaccagggaaaagtacagtttcacttccgcaatcccaacaggcgtaacttcctctttctcacggtacgtgatatcccactaacttgcaacgtcattttcccacggtcgcaccgccccttttagccgttaaccccacagccaatcaccacacgatccacactttttaaaatcacctcatttacatattggcaccattccatctataaggtatattattgatgatg